(19) United States
(12) Patent Application Publication
(10) Pub. No.: US 2015/0105409 A1
(43) Pub. Date: Apr. 16, 2015

(54) HDAC INHIBITORS, ALONE OR IN COMBINATION WITH BTK INHIBITORS, FOR TREATING NONHODGKIN'S LYMPHOMA

(71) Applicants: Steven Norman Quayle, Brookline, MA (US); Simon Stewart Jones, Harvard, MA (US); Eduardo M. Sotomayor, Tampa, FL (US); Javier Pinilla-Ibarz, Tampa, FL (US); Eva Sahakian, Tampa, FL (US)

(72) Inventors: Steven Norman Quayle, Brookline, MA (US); Simon Stewart Jones, Harvard, MA (US); Eduardo M. Sotomayor, Tampa, FL (US); Javier Pinilla-Ibarz, Tampa, FL (US); Eva Sahakian, Tampa, FL (US)

(73) Assignee: Acetylon Pharmaceticals, Inc., Boston, MA (US)

(21) Appl. No.: 14/508,135

(22) Filed: Oct. 7, 2014

Related U.S. Application Data
(60) Provisional application No. 61/889,200, filed on Oct. 10, 2013.
(60) Provisional application No. 61/911,091, filed on Dec. 3, 2013.

(51) Int. Cl. C07D 239/42; A61K 31/519; A61K 31/505

(52) U.S. Cl. CPC C07D 239/42; A61K 31/505; A61K 31/519
USPC 514/262.1; 514/275

(57) ABSTRACT

The invention relates to HDAC inhibitors, or combinations comprising an HDAC inhibitor and a BTK inhibitor for the treatment of non-hodgkin's lymphoma in a subject in need thereof. Also provided herein are methods for treating non-hodgkin's lymphoma in a subject in need thereof comprising administering to the subject a therapeutically effective amount of an HDAC inhibitor, or a combination comprising an HDAC inhibitor and a BTK inhibitor.

### CROSS-REFERENCE TO RELATED APPLICATIONS

This application claims priority to U.S. Provisional Application Ser. No. 61/889,200, filed Oct. 10, 2013, and U.S. Provisional Application Ser. No. 61/911,091, filed Dec. 3, 2013, each of which is incorporated herein by reference in its entirety.

### BACKGROUND

Histone deacetylase (HDAC) enzymes represent attractive therapeutic targets in non-hodgkin's lymphoma (NHL), but unfortunately non-selective HDAC inhibitors have led to dose-limiting toxicities in patients.

The Bruton's tyrosine kinase (BTK) inhibitors are a class of drugs that inhibit Bruton tyrosine kinase (BTK), a member of the Tec family of kinases with a very distinct role in B-cell antigen receptor (BCR) signaling.

Due to the dose-limiting toxicities of the non-selective HDAC inhibitors, there is an ongoing need in the art for more efficacious and less toxic compositions and methods for the treatment of non-hodgkin's lymphoma. In order to meet these needs, provided herein are HDAC inhibitors, pharmaceutical combinations comprising an HDAC inhibitor and a Bruton tyrosine kinase (BTK) inhibitor, and methods for the treatment of non-hodgkin's lymphoma. The compounds, combinations, and methods of the invention are well tolerated and do not exhibit the dose-limiting toxicities of prior therapies.

### SUMMARY OF THE INVENTION

Provided herein are pharmaceutical compounds for the treatment of non-hodgkin's lymphoma in a subject in need thereof. Also provided herein are pharmaceutical combinations for the treatment of non-hodgkin's lymphoma in a subject in need thereof. In addition, provided herein are methods for treating non-hodgkin's lymphoma in a subject in need thereof.

Provided in some embodiments are histone deacetylase (HDAC) inhibitors for the treatment of non-hodgkin's lymphoma in a subject in need thereof. Provided in other embodiments are combinations comprising a histone deacetylase (HDAC) inhibitor and a Bruton tyrosine kinase (BTK) inhibitor for the treatment of non-hodgkin's lymphoma in a subject in need thereof.

Provided in further embodiments are methods for treating non-hodgkin's lymphoma in a subject in need thereof comprising administering to the subject a therapeutically effective amount of a histone deacetylase (HDAC) inhibitor, or a combination comprising a histone deacetylase (HDAC) inhibitor and a Bruton tyrosine kinase (BTK) inhibitor.

In some embodiments, the compositions and combinations decrease cell viability, synergistically increase apoptosis of cells, synergistically increase cleavage of caspase 3 in cells, and synergistically arests cells in the G1/S phase of the cell cycle.

In specific embodiments, the HDAC6 specific inhibitor is a compound of Formula I:

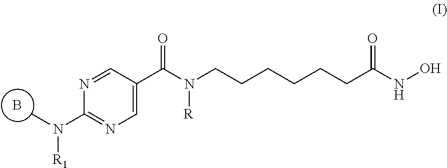

or a pharmaceutically acceptable salt thereof,
wherein,
ring B is aryl or heteroaryl;
R₁ is an aryl or heteroaryl, each of which may be optionally substituted by OH, halo, or C₁₋₆-alkyl;
and
R is H or C₁₋₆-alkyl.

In preferred embodiments, the compound of Formula I is:

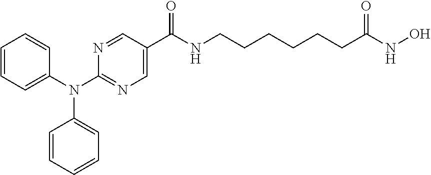 [SMILES: O=C(CCCCCCNC(=O)c1cnc(N(c2ccccc2)c2ccccc2)nc1)NO]

or a pharmaceutically acceptable salt thereof.

In yet other embodiments, the compound of Formula I is:

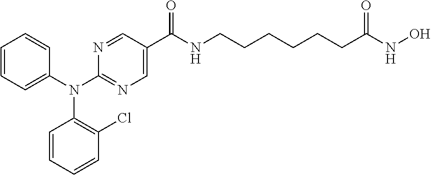 [SMILES: O=C(CCCCCCNC(=O)c1cnc(N(c2ccccc2)c2ccccc2Cl)nc1)NO]

or a pharmaceutically acceptable salt thereof.

In other specific embodiments, the HDAC6 specific inhibitor is a compound of Formula II:

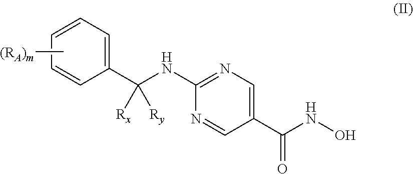

or a pharmaceutically acceptable salt thereof,
wherein,
Rₓ and R_{y} together with the carbon to which each is attached, form a cyclopropyl, cyclobutyl, cyclopentyl, cyclohexyl, cycloheptyl, or cyclooctyl;
each R_{A} is independently C₁₋₆-alkyl, C₁₋₆-alkoxy, halo, OH, —NO₂, —CN, or —NH₂; and
m is 0, 1, or 2.

In preferred embodiments, the compound of Formula II is:

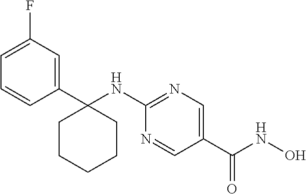 [SMILES: O=C(NO)c1cnc(NC2(c3cccc(F)c3)CCCCC2)nc1]

or a pharmaceutically acceptable salt thereof.

In other preferred embodiments, the compound of Formula II is:

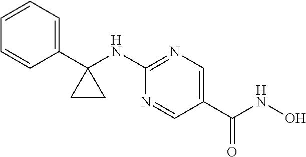 [SMILES: O=C(NO)c1cnc(NC2(c3ccccc3)CC2)nc1]

or a pharmaceutically acceptable salt thereof.

In specific embodiments, the Bruton's tyrosine kinase (BTK) inhibitor is ibrutinib or pharmaceutically acceptable salts thereof.

In some embodiments, the HDAC inhibitor is administered with a pharmaceutically acceptable carrier. In other embodiments, the combination of the HDAC inhibitor and the Bruton tyrosine kinase (BTK) inhibitor is administered with a pharmaceutically acceptable carrier.

In some embodiments, the HDAC inhibitor and the Bruton tyrosine kinase (BTK) inhibitor are administered in separate dosage forms. In other embodiments, the HDAC inhibitor and the Bruton tyrosine kinase (BTK) inhibitor are administered in a single dosage form.

In some embodiments, the HDAC inhibitor and the Bruton tyrosine kinase (BTK) inhibitor are administered at different times. In other embodiments, the HDAC inhibitor and the Bruton tyrosine kinase (BTK) inhibitor are administered at substantially the same time.

In some embodiments, the combination of the HDAC inhibitor and the Bruton tyrosine kinase (BTK) inhibitor achieves a synergistic effect in the treatment of the subject in need thereof.

In some embodiments of the combinations and/or methods, the HDAC6 specific inhibitor is a compound of Formula I:

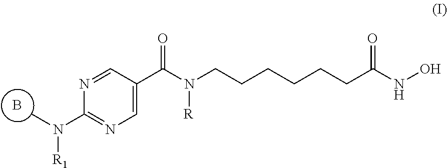

or a pharmaceutically acceptable salt thereof,
wherein,
ring B is aryl or heteroaryl;
R₁ is an aryl or heteroaryl, each of which may be optionally substituted by OH, halo, or C₁₋₆-alkyl;
and
R is H or C₁₋₆-alkyl; and

the BTK inhibitor is any BTK inhibitor.

In specific embodiments of the combinations and/or methods, the HDAC6 specific inhibitor is:

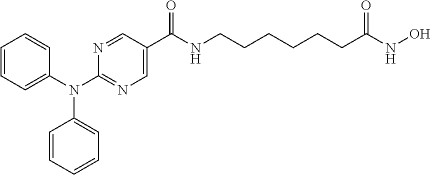 [SMILES: O=C(CCCCCCNC(=O)c1cnc(N(c2ccccc2)c2ccccc2)nc1)NO]

or a pharmaceutically acceptable salt thereof; and

the BTK inhibitor is ibrutinib or a pharmaceutically acceptable salt thereof.

In specific embodiments of the combinations and/or methods, the HDAC6 specific inhibitor is:

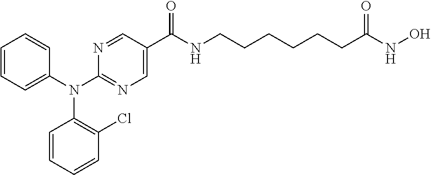 [SMILES: O=C(CCCCCCNC(=O)c1cnc(N(c2ccccc2)c2ccccc2Cl)nc1)NO]

or a pharmaceutically acceptable salt thereof; and

the BTK inhibitor is ibrutinib or a pharmaceutically acceptable salt thereof.

In some embodiments of the combinations and/or methods, the HDAC6 specific inhibitor is a compound of Formula II:

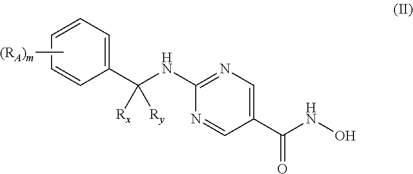

or a pharmaceutically acceptable salt thereof,
wherein,
Rₓ and R_{y} together with the carbon to which each is attached, form a cyclopropyl, cyclobutyl, cyclopentyl, cyclohexyl, cycloheptyl, or cyclooctyl;
each R_{A} is independently C₁₋₆-alkyl, C₁₋₆-alkoxy, halo, OH, —NO₂, —CN, or —NH₂; and
m is 0, 1, or 2; and

the BTK inhibitor is any BTK inhibitor.

In specific embodiments of the combinations and/or methods, the HDAC6 specific inhibitor is:

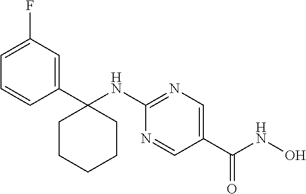 [SMILES: O=C(NO)c1cnc(NC2(c3cccc(F)c3)CCCCC2)nc1]

or a pharmaceutically acceptable salt thereof; and

the BTK inhibitor is ibrutinib or a pharmaceutically acceptable salt thereof.

In specific embodiments of the combinations and/or methods, the HDAC6 specific inhibitor is:

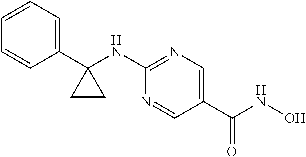 [SMILES: O=C(NO)c1cnc(NC2(c3ccccc3)CC2)nc1]

or a pharmaceutically acceptable salt thereof; and

the BTK inhibitor is ibrutinib or a pharmaceutically acceptable salt thereof.

The invention relates to methods for decreasing cell viability of cancer cells by administering an HDAC inhibitor, or a combination comprising an HDAC inhibitor and a BTK inhibitor. Preferably, the HDAC inhibitor is an HDAC6 specific inhibitor. Preferably, the BTK inhibitor is ibrutinib.

The invention also relates to methods for synergistically increasing apoptosis of cancer cells by administering a combination comprising an HDAC inhibitor and a BTK inhibitor. Preferably, the HDAC inhibitor is an HDAC6 specific inhibitor. Preferably, the BTK inhibitor is ibrutinib.

The invention further relates to methods for synergistically increasing cleavage of caspase 3 in cancer cells by administering a combination comprising an HDAC inhibitor and a BTK inhibitor. Preferably, the HDAC inhibitor is an HDAC6 specific inhibitor. Preferably, the BTK inhibitor is ibrutinib.

The invention also relates to methods for synergistically arresting cells in the G1/S phase of the cell cycle by administering a combination comprising an HDAC inhibitor and a BTK inhibitor. Preferably, the HDAC inhibitor is an HDAC6 specific inhibitor. Preferably, the BTK inhibitor is ibrutinib.

Other objects, features, and advantages will become apparent from the following detailed description. The detailed description and specific examples are given for illustration only because various changes and modifications within the spirit and scope of the invention will become apparent to those skilled in the art from this detailed description. Further, the examples demonstrate the principle of the invention.

### BRIEF DESCRIPTION OF THE FIGURES

FIGS. 1A-1E show the F_{A}/CI Synergy Plots after treatment of human lymphoma cell lines with PCI-32765 (Ibrutinib) and either Compound A or Compound C. For Mantle Cell Lymphoma (MCL) Mino, Jeko1, and Granta-519 cells were utilized, while U2932 and SUDHL16 cells represented the activated B cell (ABC) and germinal center (GC) subtypes of diffuse large B cell lymphoma (DLBCL). Data points with CI values <1 indicate treatment combinations resulting in synergistic decreases in cellular viability.

FIG. 2 shows graphs that show increased apoptosis after the treatment of MINO cells (top) and Z138 (bottom) MCL cells with DMSO, ibrutinib, Compound A, or both ibrutinib and Compound A.

FIG. 3 shows graphs that show increased apoptosis after the treatment of MINO cells (top) and Z138 (bottom) MCL cells with DMSO, ibrutinib, Compound A, or both ibrutinib and Compound A.

FIG. 4A shows graphs that show increased cleavage of Caspase 3, consistent with increased apoptosis, after the treatment of MINO cells with control, Compound A, ibrutinib, or both Compound A and ibrutinib. FIG. 4B shows graphs that show increased cleavage of Caspase 3, consistent with increased apoptosis, after the treatment of Z138 MCL cells with control, Compound A, ibrutinib, or both Compound A and ibrutinib.

FIG. 5 is a series of graphs showing that combination treatment of MCL cells with Compound A and ibrutinib resulted in decreased cell cycle progression relative to either single agent, consistent with decreased proliferation. MINO cells were treated with DMSO, ibrutinib, Compound A, or both ibrutinib and Compound A.

FIG. 6 is a graph showing the effects of treatment of CB-17 SCID mice with Vehicle, PCI-32765 (ibrutinib) alone (25 mg/kg PO QD), or PCI-32765 (25 mg/kg PO QD) plus Compound A (50 mg/kg IP QD). All treatments were well tolerated with no overt evidence of toxicity and complete recovery after minimal body weight loss.

FIG. 7 shows the effects on viability of treatment of Mec1 (top) and Wac3 (bottom) chronic lymphocytic leukemia (CLL) cells with 2 μM Compound A and 1 μM Ibrutinib (top), or 2 μM Compound A and 2 μM Ibrutinib (bottom). Combination treatment of either cell line resulted in synergistic decreases in CLL cell viability.

FIG. 8 shows the effects on viability of treatment of Z138 (top) and Mino (bottom) MCL cells with varying concentrations of Compound A and the BTKi Ibrutinib. Combination treatment of either cell line resulted in synergistic decreases in CLL cell viability.

### DETAILED DESCRIPTION

The instant application is directed, generally, to HDAC inhibitors, combinations comprising a histone deacetylase (HDAC) inhibitor and a BTK inhibitor, and methods for the treatment of non-hodgkin's lymphoma.

### DEFINITIONS

Listed below are definitions of various terms used to describe this invention. These definitions apply to the terms as they are used throughout this specification and claims, unless otherwise limited in specific instances, either individually or as part of a larger group.

The term “about” generally indicates a possible variation of no more than 10%, 5%, or 1% of a value. For example, “about 25 mg/kg” will generally indicate, in its broadest sense, a value of 22.5-27.5 mg/kg, i.e., 25±2.5 mg/kg.

The term “alkyl” refers to saturated, straight- or branched-chain hydrocarbon moieties containing, in certain embodiments, between one and six, or one and eight carbon atoms, respectively. Examples of C₁₋₆-alkyl moieties include, but are not limited to, methyl, ethyl, propyl, isopropyl, n-butyl, tert-butyl, neopentyl, n-hexyl moieties; and examples of C₁₋₈-alkyl moieties include, but are not limited to, methyl, ethyl, propyl, isopropyl, n-butyl, tert-butyl, neopentyl, n-hexyl, heptyl, and octyl moieties.

The number of carbon atoms in an alkyl substituent can be indicated by the prefix “C_{x-y},” where x is the minimum and y is the maximum number of carbon atoms in the substituent. Likewise, a Cₓ chain means an alkyl chain containing x carbon atoms.

The term “alkoxy” refers to an —O-alkyl moiety.

The terms “cycloalkyl” or “cycloalkylene” denote a monovalent group derived from a monocyclic or polycyclic saturated or partially unsaturated carbocyclic ring compound. Examples of C₃₋₈-cycloalkyl include, but are not limited to, cyclopropyl, cyclobutyl, cyclopentyl, cyclohexyl, cycloheptyl and cyclooctyl; and examples of C₃-C₁₂-cycloalkyl include, but are not limited to, cyclopropyl, cyclobutyl, cyclopentyl, cyclohexyl, bicyclo[2.2.1]heptyl, and bicyclo[2.2.2]octyl. Also contemplated are monovalent groups derived from a monocyclic or polycyclic carbocyclic ring compound having at least one carbon-carbon double bond by the removal of a single hydrogen atom. Examples of such groups include, but are not limited to, cyclopropenyl, cyclobutenyl, cyclopentenyl, cyclohexenyl, cycloheptenyl, cyclooctenyl, and the like.

The term “aryl” refers to a mono- or poly-cyclic carbocyclic ring system having one or more aromatic rings, fused or non-fused, including, but not limited to, phenyl, naphthyl, tetrahydronaphthyl, indanyl, idenyl, and the like. In some embodiments, aryl groups have 6 carbon atoms. In some embodiments, aryl groups have from six to ten carbon atoms. In some embodiments, aryl groups have from six to sixteen carbon atoms.

The term “heteroaryl” refers to a mono- or poly-cyclic (e.g., bi-, or tri-cyclic or more) fused or non-fused moiety or ring system having at least one aromatic ring, where one or more of the ring-forming atoms is a heteroatom such as oxygen, sulfur, or nitrogen. In some embodiments, the heteroaryl group has from about one to six carbon atoms, and in further embodiments from one to fifteen carbon atoms. In some embodiments, the heteroaryl group contains five to sixteen ring atoms of which one ring atom is selected from oxygen, sulfur, and nitrogen; zero, one, two, or three ring atoms are additional heteroatoms independently selected from oxygen, sulfur, and nitrogen; and the remaining ring atoms are carbon. Heteroaryl includes, but is not limited to, pyridinyl, pyrazinyl, pyrimidinyl, pyrrolyl, pyrazolyl, imidazolyl, thiazolyl, oxazolyl, isooxazolyl, thiazolyl, thiadiazolyl, oxadiazolyl, thiophenyl, furanyl, indolyl, quinolinyl, isoquinolinyl, benzimidazolyl, benzooxazolyl, quinoxalinyl, acridinyl, and the like.

The term “halo” refers to a halogen, such as fluorine, chlorine, bromine, and iodine.

The term “combination” refers to two or more therapeutic agents to treat a therapeutic condition or disorder described in the present disclosure. Such combination of therapeutic agents may be in the form of a single pill, capsule, or intravenous solution. However, the term “combination” also encompasses the situation when the two or more therapeutic agents are in separate pills, capsules, or intravenous solutions. Likewise, the term “combination therapy” refers to the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, or in separate containers (e.g., capsules) for each active ingredient. In addition, such administration also encompasses use of each type of therapeutic agent in a sequential manner, either at approximately the same time or at different times. In either case, the treatment regimen will provide beneficial effects of the drug combination in treating the conditions or disorders described herein.

The term “HDAC” refers to histone deacetylases, which are enzymes that remove the acetyl groups from the lysine residues in core histones, thus leading to the formation of a condensed and transcriptionally silenced chromatin. There are currently 18 known histone deacetylases, which are classified into four groups. Class I HDACs, which include HDAC1, HDAC2, HDAC3, and HDAC8, are related to the yeast RPD3 gene. Class II HDACs, which include HDAC4, HDAC5, HDAC6, HDAC7, HDAC9, and HDAC10, are related to the yeast Hdal gene. Class III HDACs, which are also known as the sirtuins are related to the Sir2 gene and include SIRT1-7. Class IV HDACs, which contains only HDAC11, has features of both Class I and II HDACs. The term “HDAC” refers to any one or more of the 18 known histone deacetylases, unless otherwise specified.

The term “HDAC6 specific” means that the compound binds to HDAC6 to a substantially greater extent, such as 5×, 10×, 15×, 20× greater or more, than to any other type of HDAC enzyme, such as HDAC1 or HDAC2. That is, the compound is selective for HDAC6 over any other type of HDAC enzyme. For example, a compound that binds to HDAC6 with an IC₅₀ of 10 nM and to HDAC1 with an IC₅₀ of 50 nM is HDAC6 specific. On the other hand, a compound that binds to HDAC6 with an IC₅₀ of 50 nM and to HDAC1 with an IC₅₀ of 60 nM is not HDAC6 specific

The term “inhibitor” is synonymous with the term antagonist.

### Histone Deacetylase (HDAC) Inhibitors

Provided herein are compounds and pharmaceutical combinations for the treatment of non-hodgkin's lymphoma in a subject in need thereof. Also provided herein are methods for treating non-hodgkin's lymphoma in a subject in need thereof.

The compounds, combinations, and methods of the invention comprise a histone deacetylase (HDAC) inhibitor. The HDAC inhibitor may be any HDAC inhibitor. Thus, the HDAC inhibitor may be selective or non-selective to a particular type of histone deacetylase enzyme. Preferably, the HDAC inhibitor is a selective HDAC inhibitor. More preferably, the HDAC inhibitor is an HDAC6 specific inhibitor.

In some embodiments, the HDAC6 specific inhibitor is a compound of Formula I:

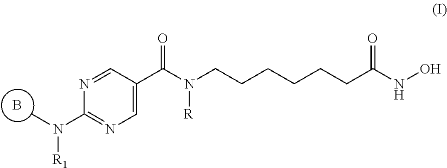

or a pharmaceutically acceptable salt thereof,
wherein,
ring B is aryl or heteroaryl;
R₁ is an aryl or heteroaryl, each of which may be optionally substituted by OH, halo, or C₁₋₆-alkyl;
and
R is H or C₁₋₆-alkyl.

Representative compounds of Formula I include, but are not limited to:

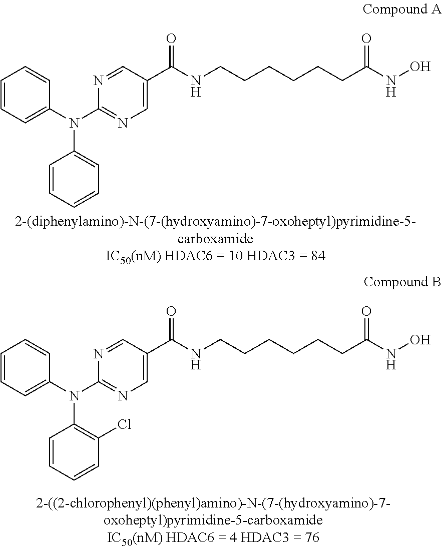 [SMILES: O=C(CCCCCCNC(=O)c1cnc(N(c2ccccc2)c2ccccc2)nc1)NO ; O=C(CCCCCCNC(=O)c1cnc(N(c2ccccc2)c2ccccc2Cl)nc1)NO]

or pharmaceutically acceptable salts thereof.

The preparation and properties of selective HDAC6 inhibitors according to Formula I are provided in International Patent Application No. PCT/US2011/021982, the entire contents of which are incorporated herein by reference.

In other embodiments, the HDAC6 specific inhibitor is a compound of Formula II:

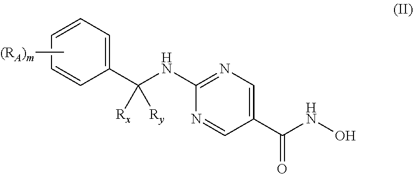

or a pharmaceutically acceptable salt thereof,
wherein,
Rₓ and R_{y} together with the carbon to which each is attached, form a cyclopropyl, cyclobutyl, cyclopentyl, cyclohexyl, cycloheptyl, or cyclooctyl;
each R_{A} is independently C₁₋₆-alkyl, C₁₋₆-alkoxy, halo, OH, —NO₂, —CN, or —NH₂; and
m is 0, 1, or 2.

Representative compounds of Formula II include, but are not limited to:

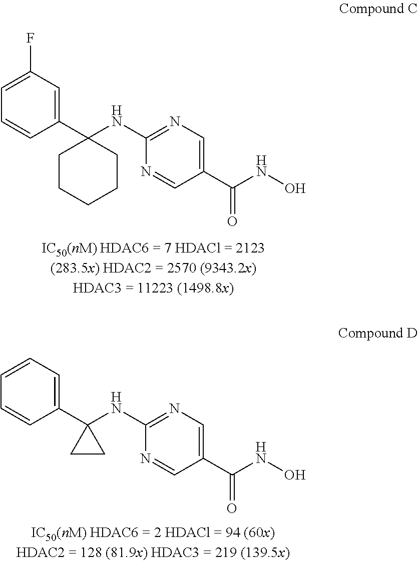 [SMILES: O=C(NO)c1cnc(NC2(c3cccc(F)c3)CCCCC2)nc1 ; O=C(NO)c1cnc(NC2(c3ccccc3)CC2)nc1]

or pharmaceutically acceptable salts thereof.

The preparation and properties of selective HDAC6 inhibitors according to Formula II are provided in International Patent Application No. PCT/US2011/060791, the entire contents of which are incorporated herein by reference.

In some embodiments, the compounds described herein are unsolvated. In other embodiments, one or more of the compounds are in solvated form. As known in the art, the solvate can be any of pharmaceutically acceptable solvent, such as water, ethanol, and the like.

### Bruton's Tyrosine Kinase (BTK) Inhibitor

The combinations of the invention comprise a BTK inhibitor. Some embodiments of the methods also comprise a BTK inhibitor. The BTK inhibitor may be any BTK inhibitor. Preferably, the BTK inhibitor is ibrutinib.

The terms “Bruton's tyrosine kinase inhibitor” and “BTK inhibitor” refer to any compound that reduces a catalytic activity of Bruton's tyrosine kinase (BTK), or homolog thereof, and thereby reduces BTK-mediated signaling.

The term “Bruton's tyrosine kinase (BTK)” refers to Bruton's tyrosine kinase from Homo sapiens, as disclosed in, e.g., U.S. Pat. No. 6,326,469 (GenBank Accession No. NP-000052), or a homolog thereof.

The term “Bruton's tyrosine kinase homolog” refers to orthologs of Bruton's tyrosine kinase, e.g., the orthologs from mouse (GenBank Accession No. AAB47246), dog (GenBank Accession No. XP-549139), rat (GenBank Accession No. NP-001007799), chicken (GenBank Accession No. NP-989564), or zebra fish (GenBank Accession No. XP-698117), and fusion proteins of any of the foregoing that exhibit kinase activity towards one or more substrates of Bruton's tyrosine kinase.

The phrase “BTK-mediated signaling” means any of the biological activities that are dependent on, either directly or indirectly, the activity of BTK. Examples of BTK-mediated signaling are signals that lead to proliferation and survival of BTK-expressing cells, and stimulation of one or more BTK-signaling pathways within BTK-expressing cells.

A BTK “signaling pathway” or “signal transduction pathway” is intended to mean at least one biochemical reaction, or a group of biochemical reactions, that results from the activity of BTK, and which generates a signal that, when transmitted through the signal pathway, leads to activation of one or more downstream molecules in the signaling cascade. Signal transduction pathways involve a number of signal transduction molecules that lead to transmission of a signal from the cell-surface across the plasma membrane of a cell, and through one or more in a series of signal transduction molecules, through the cytoplasm of the cell, and in some instances, into the cell's nucleus. Of particular interest to the present invention are BTK signal transduction pathways which ultimately regulate (either enhance or inhibit) the activation of NF-κB . . . via the NF-κB signaling pathway.

In some embodiments, a BTK inhibitor can be an antagonist anti-BTK antibody. In one embodiment of the invention, the antagonist anti-BTK antibody is free of significant agonist activity in one cellular response. In another embodiment of the invention, the antagonist anti-BTK antibody is free of significant agonist activity in assays of more than one cellular response (e.g., proliferation and differentiation, or proliferation, differentiation, and, for B cells, antibody production).

In other embodiments, the BTK inhibitor can be either a reversible or irreversible small molecule inhibitor (recently reviewed by D'Cruz et al., OncoTargets and Therapy 2013:6 161-176).

The term “irreversible BTK inhibitor” refers to an inhibitor of BTK that can form a covalent bond with an amino acid residue of BTK that results in a reduction of BTK signaling activity. In one embodiment, the irreversible inhibitor of BTK can form a covalent bond with a Cys residue of BTK; in particular embodiments, the irreversible inhibitor can form a covalent bond with a Cys 481 residue (or a homolog thereof) of BTK. Examples of irreversible BTK inhibitors include, but are not limited to, for example, ibrutinib/PCI-32765 (see structure below and U.S. Pat. No. 8,088,781), CNX-774, CC-292, AVL-101, and AVL-291/292.

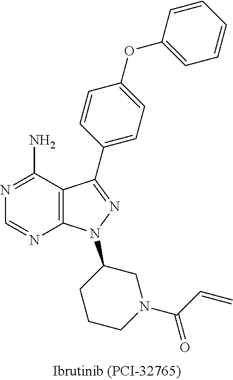 [SMILES: C=CC(=O)N1CCC[C@@H](n2nc(-c3ccc(Oc4ccccc4)cc3)c3c(N)ncnc32)C1]

The term “reversible BTK inhibitor” refers to an inhibitor of BTK that reversibly binds to BTK to reduce BTK signaling activity. Examples of reversible BTK inhibitors include, but are not limited to Dasatinib (Sprycel/BMS-354825, Bristol-Myers Squibb) [N-(2-chloro-6-methylphenyl)-2-(6-(4-(2-hydroxyethyl)piperazin-1-yl)-2-methylpyrimidin-4-ylamino)thiazole5-carboxamide], LFM-A13, ONO-WG-307, RN-486, GDC-0834.

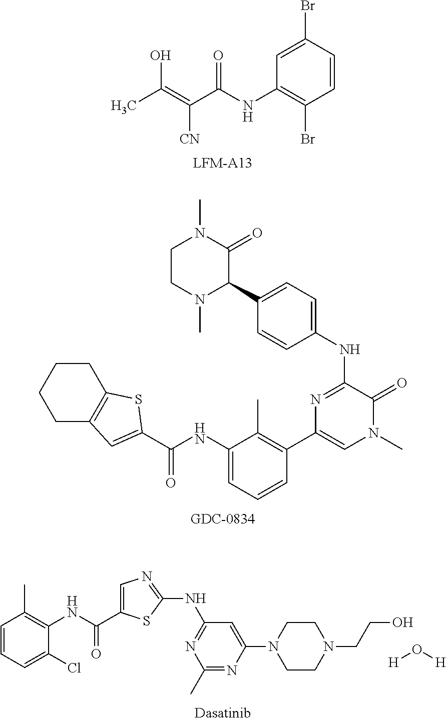

BTK inhibitors currently in clinical development are reviewed by Akinleye et al. Journal of Hematology & Oncology 2013, 6:59.

In some embodiments, the compounds described herein are unsolvated. In other embodiments, one or more of the compounds are in solvated form. As known in the art, the solvate can be any of pharmaceutically acceptable solvent, such as water, ethanol, and the like.

### Compositions, Combinations, and Pharmaceutical Compositions and Combinations

Provided herein are compositions and combinations for the treatment of non-hodgkin's lymphoma in a subject in need thereof. Provided in some embodiments are HDAC inhibitors, or combinations comprising a histone deacetylase (HDAC) inhibitor and a Bruton's tyrosine kinase (BTK) inhibitor for the treatment of non-hodgkin's lymphoma in a subject in need thereof. Provided in some embodiments are HDAC inhibitors, or combinations comprising a histone deacetylase (HDAC) inhibitor and a Bruton's tyrosine kinase (BTK) inhibitor for the treatment of non-hodgkin's lymphoma in a subject in need thereof, wherein the combination is administered at dosages that would not be effective when one or both of the compounds are administered alone, but which amounts are effective in combination.

In some embodiments of the compositions and combinations, the HDAC inhibitor is an HDAC6 specific inhibitor. In specific embodiments, the HDAC6 specific inhibitor is a compound of Formula I:

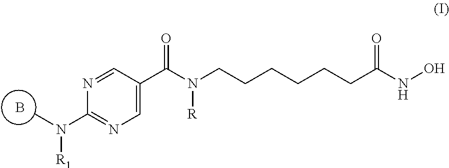

or a pharmaceutically acceptable salt thereof.

In preferred embodiments, the compound of Formula I is:

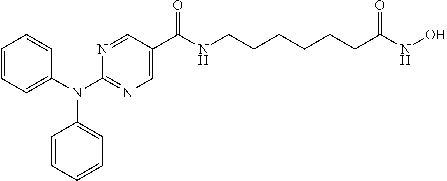 [SMILES: O=C(CCCCCCNC(=O)c1cnc(N(c2ccccc2)c2ccccc2)nc1)NO]

or a pharmaceutically acceptable salt thereof.

In yet other embodiments, the compound of Formula I is:

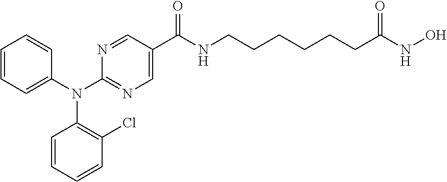 [SMILES: O=C(CCCCCCNC(=O)c1cnc(N(c2ccccc2)c2ccccc2Cl)nc1)NO]

or a pharmaceutically acceptable salt thereof.

In other specific embodiments, the HDAC6 specific inhibitor is a compound of Formula II:

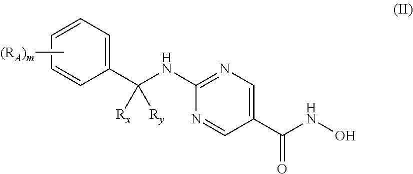

or a pharmaceutically acceptable salt thereof.

In preferred embodiments, the compound of Formula II is:

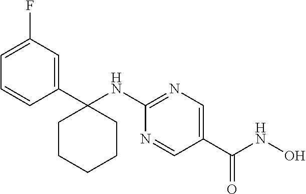 [SMILES: O=C(NO)c1cnc(NC2(c3cccc(F)c3)CCCCC2)nc1]

or a pharmaceutically acceptable salt thereof.

In other preferred embodiments, the compound of Formula II is:

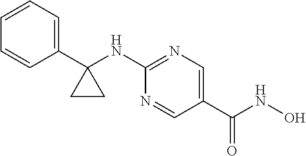 [SMILES: O=C(NO)c1cnc(NC2(c3ccccc3)CC2)nc1]

or a pharmaceutically acceptable salt thereof.

In some embodiments of the combinations, the Bruton's tyrosine kinase (BTK) inhibitor is ibrutinib:

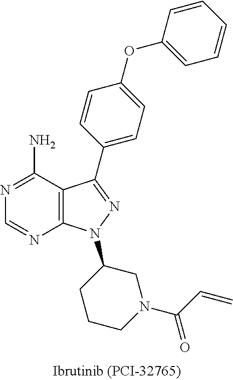 [SMILES: C=CC(=O)N1CCC[C@@H](n2nc(-c3ccc(Oc4ccccc4)cc3)c3c(N)ncnc32)C1]

or a pharmaceutically acceptable salt thereof.

In one embodiment, provided herein is a combination therapy comprising an HDAC6 specific inhibitor and a Bruton's tyrosine kinase (BTK) inhibitor, wherein the HDAC6 specific inhibitor is a compound of Formula I:

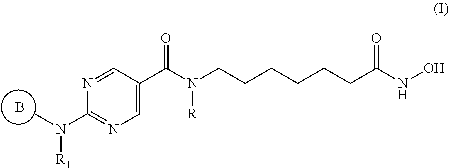

or a pharmaceutically acceptable salt thereof,
wherein,
ring B is aryl or heteroaryl;
R₁ is an aryl or heteroaryl, each of which may be optionally substituted by OH, halo, or C₁₋₆-alkyl;
and
R is H or C₁₋₆-alkyl; and
the BTK inhibitor is any BTK inhibitor.

In specific embodiments of the combinations, the HDAC6 specific inhibitor is:

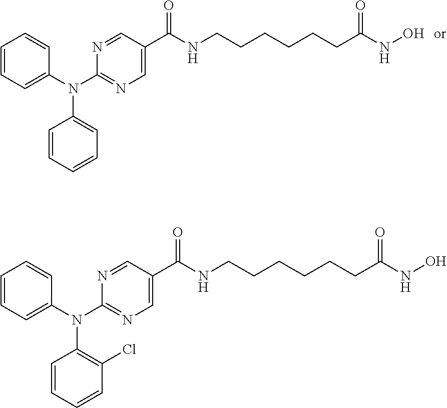 [SMILES: O=C(CCCCCCNC(=O)c1cnc(N(c2ccccc2)c2ccccc2)nc1)NO ; O=C(CCCCCCNC(=O)c1cnc(N(c2ccccc2)c2ccccc2Cl)nc1)NO]

or pharmaceutically acceptable salts thereof; and
the BTK inhibitor is ibrutinib or a pharmaceutically acceptable salt thereof.

In other embodiments, provided herein is a combination therapy comprising an HDAC6 specific inhibitor and a Bruton's tyrosine kinase (BTK) inhibitor, wherein the HDAC6 specific inhibitor is a compound of Formula II:

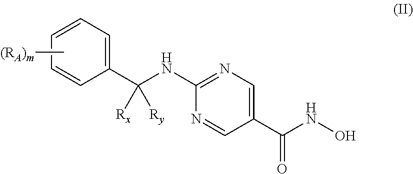

or a pharmaceutically acceptable salt thereof,
wherein,
Rₓ and R_{y} together with the carbon to which each is attached, form a cyclopropyl, cyclobutyl, cyclopentyl, cyclohexyl, cycloheptyl, or cyclooctyl;
each R_{A} is independently C₁₋₆-alkyl, C₁₋₆-alkoxy, halo, OH, —NO₂, —CN, or —NH₂; and
m is 0, 1, or 2; and
the BTK inhibitor any BTK inhibitor.

In specific embodiments of the combinations, the HDAC6 specific inhibitor is:

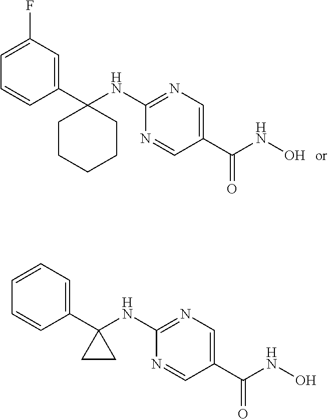 [SMILES: O=C(NO)c1cnc(NC2(c3cccc(F)c3)CCCCC2)nc1 ; O=C(NO)c1cnc(NC2(c3ccccc3)CC2)nc1]

or a pharmaceutically acceptable salt thereof; and

the BTK inhibitor is ibrutinib or a pharmaceutically acceptable salt thereof.

Although the compounds of Formulas I and II are depicted in their neutral forms, in some embodiments, these compounds are used in a pharmaceutically acceptable salt form. “Pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the conventional non-toxic salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17.sup.th ed., Mack Publishing Company, Easton, Pa., 1985, p. 1418 and Journal of Pharmaceutical Science, 66, 2 (1977), each of which is incorporated herein by reference in its entirety.

### Administration/Dose

In some embodiments, the HDAC inhibitor (a compound of Formula I or II) is administered simultaneously with the Bruton's tyrosine kinase (BTK) inhibitor. Simultaneous administration typically means that both compounds enter the patient at precisely the same time. However, simultaneous administration also includes the possibility that the HDAC inhibitor and the Bruton's tyrosine kinase (BTK) inhibitor enter the patient at different times, but the difference in time is sufficiently miniscule that the first administered compound is not provided the time to take effect on the patient before entry of the second administered compound. Such delayed times typically correspond to less than 1 minute, and more typically, less than 30 seconds. In one example, wherein the compounds are in solution, simultaneous administration can be achieved by administering a solution containing the combination of compounds. In another example, simultaneous administration of separate solutions, one of which contains the HDAC inhibitor and the other of which contains the Bruton's tyrosine kinase (BTK) inhibitor, can be employed. In one example wherein the compounds are in solid form, simultaneous administration can be achieved by administering a composition containing the combination of compounds. Alternatively, simultaneous administration can be achieved by administering two separate compositions, one comprising the HDAC inhibitor and the other comprising the Bruton's tyrosine kinase (BTK) inhibitor.

In other embodiments, the HDAC inhibitor and the Bruton's tyrosine kinase (BTK) inhibitor are not administered simultaneously. In some embodiments, the HDAC inhibitor is administered before the Bruton's tyrosine kinase (BTK) inhibitor. In other embodiments, the Bruton's tyrosine kinase (BTK) inhibitor is administered before the HDAC inhibitor. The time difference in non-simultaneous administrations can be greater than 1 minute, five minutes, 10 minutes, 15 minutes, 30 minutes, 45 minutes, 60 minutes, two hours, three hours, six hours, nine hours, 12 hours, etc. In other embodiments, the first administered compound is provided time to take effect on the patient before the second administered compound is administered. Generally, the difference in time does not extend beyond the time for the first administered compound to complete its effect in the patient, or beyond the time the first administered compound is completely or substantially eliminated or deactivated in the patient.

In some embodiments, one or both of the HDAC inhibitor and the Bruton's tyrosine kinase (BTK) inhibitor are administered in a therapeutically effective amount or dosage. A “therapeutically effective amount” is an amount of HDAC6 specific inhibitor (a compound of Formula I or II) or a Bruton's tyrosine kinase (BTK) inhibitor that, when administered to a patient by itself, effectively treats the non-hodgkin's lymphoma. An amount that proves to be a “therapeutically effective amount” in a given instance, for a particular subject, may not be effective for 100% of subjects similarly treated for the disease or condition under consideration, even though such dosage is deemed a “therapeutically effective amount” by skilled practitioners. The amount of the compound that corresponds to a therapeutically effective amount is strongly dependent on the type of cancer, stage of the cancer, the age of the patient being treated, and other facts. In general, therapeutically effective amounts of these compounds are well-known in the art, such as provided in the supporting references cited above.

In other embodiments, one or both of the HDAC inhibitor and the Bruton's tyrosine kinase (BTK) inhibitor are administered in a sub-therapeutically effective amount or dosage. A sub-therapeutically effective amount is an amount of HDAC inhibitor (for example, a compound of Formula I or II) or a Bruton's tyrosine kinase (BTK) inhibitor that, when administered to a patient by itself, does not completely inhibit over time the biological activity of the intended target.

Whether administered in therapeutic or sub-therapeutic amounts, the combination of the HDAC inhibitor and the Bruton's tyrosine kinase (BTK) inhibitor should be effective in treating non-hodgkin's lymphoma. For example, a sub-therapeutic amount of the Bruton's tyrosine kinase (BTK) inhibitor can be an effective amount if, when combined with a compound a compound of Formula I or II (HDAC6 specific inhibitor), the combination is effective in the treatment of non-hodgkin's lymphoma.

In some embodiments, the combination of compounds exhibits a synergistic effect (i.e., greater than additive effect) in the treatment of the non-hodgkin's lymphoma. The term “synergistic effect” refers to the action of two agents, such as, for example, a HDAC inhibitor and a Bruton's tyrosine kinase (BTK) inhibitor, producing an effect, for example, slowing the symptomatic progression of cancer or symptoms thereof, which is greater than the simple addition of the effects of each drug administered by themselves. A synergistic effect can be calculated, for example, using suitable methods such as the Sigmoid-Emax equation (Holford, N. H. G. and Scheiner, L. B., Clin. Pharmacokinet. 6: 429-453 (1981)), the equation of Loewe additivity (Loewe, S, and Muischnek, H., Arch. Exp. Pathol Pharmacol. 114: 313-326 (1926)) and the median-effect equation (Chou, T. C. and Talalay, P., Adv. Enzyme Regul. 22: 27-55 (1984)). Each equation referred to above can be applied to experimental data to generate a corresponding graph to aid in assessing the effects of the drug combination. The corresponding graphs associated with the equations referred to above are the concentration-effect curve, isobologram curve and combination index curve, respectively.

In different embodiments, depending on the combination and the effective amounts used, the combination of compounds can inhibit cancer growth, achieve cancer stasis, or even achieve substantial or complete cancer regression.

While the amounts of a HDAC inhibitor and a Bruton's tyrosine kinase (BTK) inhibitor should result in the effective treatment of the non-hodgkin's lymphoma, the amounts, when combined, are preferably not excessively toxic to the patient (i.e., the amounts are preferably within toxicity limits as established by medical guidelines). In some embodiments, either to prevent excessive toxicity and/or provide a more efficacious treatment of the non-hodgkin's lymphoma, a limitation on the total administered dosage is provided. Typically, the amounts considered herein are per day; however, half-day and two-day or three-day cycles also are considered herein.

Different dosage regimens may be used to treat the non-hodgkin's lymphoma. In some embodiments, a daily dosage, such as any of the exemplary dosages described above, is administered once, twice, three times, or four times a day for three, four, five, six, seven, eight, nine, or ten days. Depending on the stage and severity of the non-hodgkin's lymphoma, a shorter treatment time (e.g., up to five days) may be employed along with a high dosage, or a longer treatment time (e.g., ten or more days, or weeks, or a month, or longer) may be employed along with a low dosage. In some embodiments, a once- or twice-daily dosage is administered every other day.

In some embodiments, each dosage contains both an HDAC inhibitor and an Bruton's tyrosine kinase (BTK) inhibitor to be delivered as a single dosage, while in other embodiments, each dosage contains either a HDAC inhibitor and a Bruton's tyrosine kinase (BTK) inhibitor to be delivered as separate dosages.

Compounds of Formula I or II, or their pharmaceutically acceptable salts or solvate forms, in pure form or in an appropriate pharmaceutical composition, can be administered via any of the accepted modes of administration or agents known in the art. The compounds can be administered, for example, orally, nasally, parenterally (intravenous, intramuscular, or subcutaneous), topically, transdermally, intravaginally, intravesically, intracistemally, or rectally. The dosage form can be, for example, a solid, semi-solid, lyophilized powder, or liquid dosage forms, such as for example, tablets, pills, soft elastic or hard gelatin capsules, powders, solutions, suspensions, suppositories, aerosols, or the like, preferably in unit dosage forms suitable for simple administration of precise dosages. A particular route of administration is oral, particularly one in which a convenient daily dosage regimen can be adjusted according to the degree of severity of the disease to be treated.

As discussed above, the HDAC inhibitor and the BTK inhibitor of the pharmaceutical combination can be administered in a single unit dose or separate dosage forms. Accordingly, the phrase “pharmaceutical combination” includes a combination of two drugs in either a single dosage form or a separate dosage forms, i.e., the pharmaceutically acceptable carriers and excipients described throughout the application can be combined with an HDAC inhibitor and a BTK inhibitor in a single unit dose, as well as individually combined with a HDAC inhibitor and a BTK inhibitor when these compounds are administered separately.

Auxiliary and adjuvant agents may include, for example, preserving, wetting, suspending, sweetening, flavoring, perfuming, emulsifying, and dispensing agents. Prevention of the action of microorganisms is generally provided by various antibacterial and antifungal agents, such as, parabens, chlorobutanol, phenol, sorbic acid, and the like. Isotonic agents, such as sugars, sodium chloride, and the like, may also be included. Prolonged absorption of an injectable pharmaceutical form can be brought about by the use of agents delaying absorption, for example, aluminum monostearate and gelatin. The auxiliary agents also can include wetting agents, emulsifying agents, pH buffering agents, and antioxidants, such as, for example, citric acid, sorbitan monolaurate, triethanolamine oleate, butylated hydroxytoluene, and the like.

Solid dosage forms can be prepared with coatings and shells, such as enteric coatings and others well-known in the art. They can contain pacifying agents and can be of such composition that they release the active compound or compounds in a certain part of the intestinal tract in a delayed manner. Examples of embedded compositions that can be used are polymeric substances and waxes. The active compounds also can be in microencapsulated form, if appropriate, with one or more of the above-mentioned excipients.

Liquid dosage forms for oral administration include pharmaceutically acceptable emulsions, solutions, suspensions, syrups, and elixirs. Such dosage forms are prepared, for example, by dissolving, dispersing, etc., the HDAC inhibitors or Bruton's tyrosine kinase (BTK) inhibitors described herein, or a pharmaceutically acceptable salt thereof, and optional pharmaceutical adjuvants in a carrier, such as, for example, water, saline, aqueous dextrose, glycerol, ethanol and the like; solubilizing agents and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethyl formamide; oils, in particular, cottonseed oil, groundnut oil, corn germ oil, olive oil, castor oil and sesame oil, glycerol, tetrahydrofurfuryl alcohol, polyethyleneglycols and fatty acid esters of sorbitan; or mixtures of these substances, and the like, to thereby form a solution or suspension.

Generally, depending on the intended mode of administration, the pharmaceutically acceptable compositions will contain about 1% to about 99% by weight of the compounds described herein, or a pharmaceutically acceptable salt thereof, and 99% to 1% by weight of a pharmaceutically acceptable excipient. In one example, the composition will be between about 5% and about 75% by weight of a compound described herein, or a pharmaceutically acceptable salt thereof, with the rest being suitable pharmaceutical excipients.

Actual methods of preparing such dosage forms are known, or will be apparent, to those skilled in this art. Reference is made, for example, to Remington's Pharmaceutical Sciences, 18th Ed., (Mack Publishing Company, Easton, Pa., 1990).

### Methods of the Invention

The invention relates to methods for treating non-hodgkin's lymphoma in a subject in need thereof comprising administering to the subject an HDAC inhibitor, or a pharmaceutical combination of the invention. Thus, provided herein are methods for treating non-hodgkin's lymphoma in a subject in need thereof comprising administering to the subject a therapeutically effective amount of an HDAC inhibitor, or a combination comprising an HDAC inhibitor and a Bruton's tyrosine kinase (BTK) inhibitor.

The subject considered herein is typically a human. However, the subject can be any mammal for which treatment is desired. Thus, the methods described herein can be applied to both human and veterinary applications.

The terms “treating” or “treatment” indicate that the method has, at the least, mitigated abnormal cellular proliferation. For example, the method can reduce the rate of cellular growth in a patient, or prevent the continued growth or spread of non-hodgkin's lymphoma, or even reduce the overall reach of the non-hodgkin's lymphoma. Inhibition of abnormal cell growth can occur by a variety of mechanisms including, but not limited to, cell death, apoptosis, arrest of mitosis, inhibition of cell division, transcription, translation, transduction, etc.

As such, in one embodiment, provided herein is a method for treating non-hodgkin's lymphoma in a subject in need thereof comprising administering to the subject a therapeutically effective amount of Compound A.

In another embodiment is a method for treating non-hodgkin's lymphoma in a subject in need thereof comprising administering to the subject a therapeutically effective amount of Compound B.

In another embodiment is a method for treating non-hodgkin's lymphoma in a subject in need thereof comprising administering to the subject a therapeutically effective amount of Compound C.

In another embodiment is a method for treating non-hodgkin's lymphoma in a subject in need thereof comprising administering to the subject a therapeutically effective amount of Compound D.

In another embodiment is a method for treating non-hodgkin's lymphoma in a subject in need thereof comprising administering to the subject a therapeutically effective amount of Compound A and ibrutinib.

In another embodiment is a method for treating non-hodgkin's lymphoma in a subject in need thereof comprising administering to the subject a therapeutically effective amount of Compound B and ibrutinib.

In another embodiment is a method for treating non-hodgkin's lymphoma in a subject in need thereof comprising administering to the subject a therapeutically effective amount of Compound C and ibrutinib.

In another embodiment is a method for treating non-hodgkin's lymphoma in a subject in need thereof comprising administering to the subject a therapeutically effective amount of Compound D and ibrutinib.

The invention relates to methods for decreasing cell viability of cancer cells by administering an HDAC inhibitor, or a combination comprising an HDAC inhibitor and a BTK inhibitor. Preferably, the HDAC inhibitor is an HDAC6 specific inhibitor. Preferably, the BTK inhibitor is ibrutinib.

The invention also relates to methods for synergistically increasing apoptosis of cancer cells by administering a combination comprising an HDAC inhibitor and a BTK inhibitor. Preferably, the HDAC inhibitor is an HDAC6 specific inhibitor. Preferably, the BTK inhibitor is ibrutinib.

The invention further relates to methods for synergistically increasing cleavage of caspase 3 in cancer cells by administering a combination comprising an HDAC inhibitor and a BTK inhibitor. Preferably, the HDAC inhibitor is an HDAC6 specific inhibitor. Preferably, the BTK inhibitor is ibrutinib.

The invention also relates to methods for synergistically arresting cells in the G1/S phase of the cell cycle by administering a combination comprising an HDAC inhibitor and a BTK inhibitor. Preferably, the HDAC inhibitor is an HDAC6 specific inhibitor. Preferably, the BTK inhibitor is ibrutinib.

### Kits

In other embodiments, kits are provided. Kits according to the invention include package(s) comprising compounds or compositions of the invention. In some embodiments, kits comprise a HDAC inhibitor or a pharmaceutically acceptable salt thereof, or a HDAC inhibitor or a pharmaceutically acceptable salt thereof and a Bruton's tyrosine kinase (BTK) inhibitor or a pharmaceutically acceptable salt thereof.

The phrase “package” means any vessel containing compounds or compositions presented herein. In some embodiments, the package can be a box or wrapping. Packaging materials for use in packaging pharmaceutical products are well-known to those of skill in the art. Examples of pharmaceutical packaging materials include, but are not limited to, bottles, tubes, inhalers, pumps, bags, vials, containers, syringes, bottles, and any packaging material suitable for a selected formulation and intended mode of administration and treatment.

The kit can also contain items that are not contained within the package, but are attached to the outside of the package, for example, pipettes.

Kits can further contain instructions for administering compounds or compositions of the invention to a patient. Kits also can comprise instructions for approved uses of compounds herein by regulatory agencies, such as the United States Food and Drug Administration. Kits can also contain labeling or product inserts for the compounds. The package(s) and/or any product insert(s) may themselves be approved by regulatory agencies. The kits can include compounds in the solid phase or in a liquid phase (such as buffers provided) in a package. The kits can also include buffers for preparing solutions for conducting the methods, and pipettes for transferring liquids from one container to another.

### EXAMPLES

Examples have been set forth below for the purpose of illustration and to describe certain specific embodiments of the invention. However, the scope of the claims is not to be in any way limited by the examples set forth herein. Various changes and modifications to the disclosed embodiments will be apparent to those skilled in the art and such changes and modifications including, without limitation, those relating to the chemical structures, substituents, derivatives, formulations and/or methods of the invention may be made without departing from the spirit of the invention and the scope of the appended claims. Definitions of the variables in the structures in the schemes herein are commensurate with those of corresponding positions in the formulae presented herein.

The synthesis of the compounds of Formula I (e.g., Compounds A and B) is provided in PCT/US2011/021982, which is incorporated herein by reference in its entirety. The synthesis of compounds of Formula II (e.g., Compounds C and D) is provided in PCT/US2011/060791, which is incorporated herein by reference in its entirety.

### Example 1

### Synthesis of 2-(diphenylamino)-N-(7-(hydroxyamino)-7-oxoheptyl) pyrimidine-5-carboxamide (Compound A)

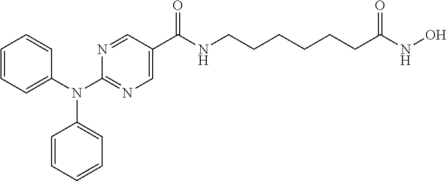 [SMILES: O=C(CCCCCCNC(=O)c1cnc(N(c2ccccc2)c2ccccc2)nc1)NO]

### Reaction Scheme

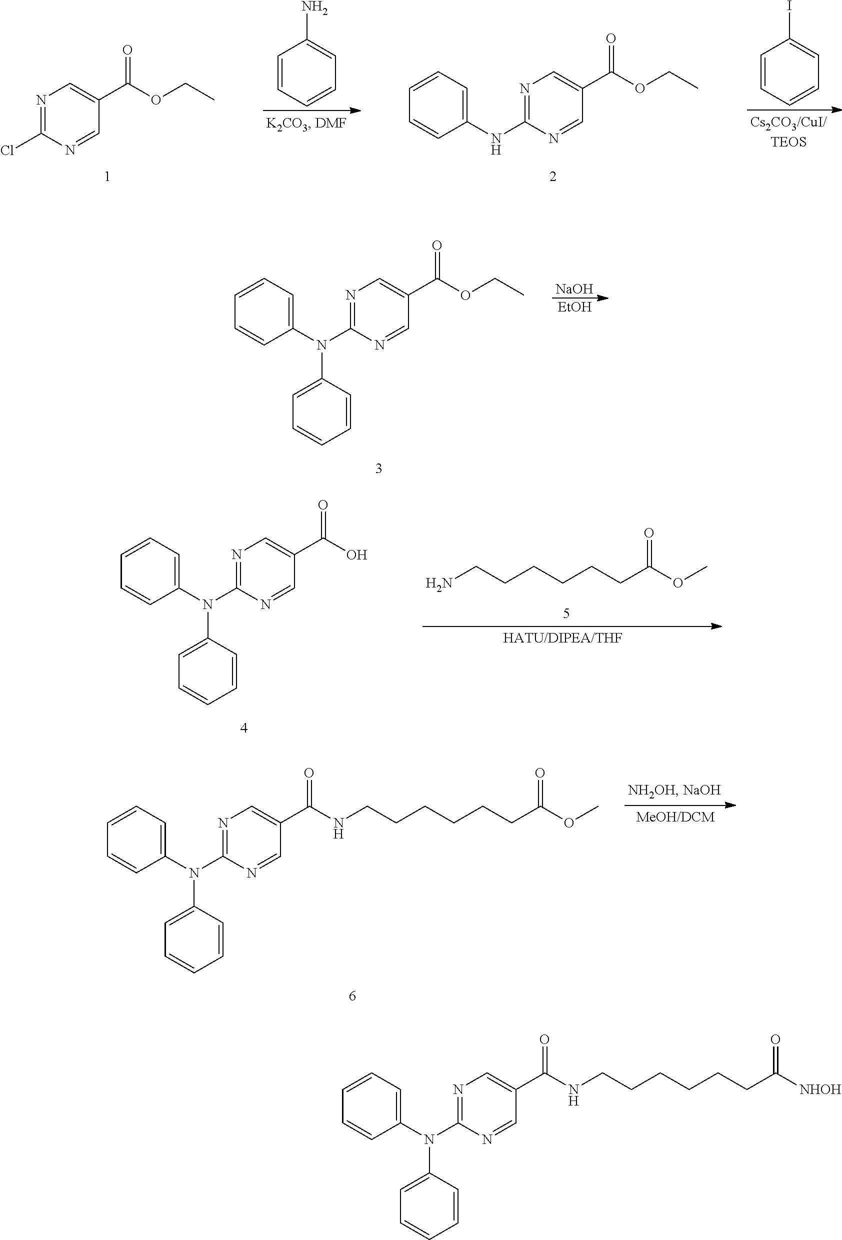

### Synthesis of Intermediate 2

A mixture of aniline (3.7 g, 40 mmol), compound 1 (7.5 g, 40 mmol), and K₂CO₃ (11 g, 80 mmol) in DMF (100 ml) was degassed and stirred at 120° C. under N₂ overnight. The reaction mixture was cooled to r.t. and diluted with EtOAc (200 ml), then washed with saturated brine (200 ml×3). The organic layers were separated and dried over Na₂SO₄, evaporated to dryness and purified by silica gel chromatography (petroleum ethers/EtOAc=10/1) to give the desired product as a white solid (6.2 g, 64%).

### Synthesis of Intermediate 3

A mixture of compound 2 (6.2 g, 25 mmol), iodobenzene (6.12 g, 30 mmol), CuI (955 mg, 5.0 mmol), Cs₂CO₃ (16.3 g, 50 mmol) in TEOS (200 ml) was degassed and purged with nitrogen. The resulting mixture was stirred at 140° C. for 14 hrs. After cooling to r.t., the residue was diluted with EtOAc (200 ml). 95% EtOH (200 ml) and NH₄F—H₂O on silica gel [50 g, pre-prepared by the addition of NH₄F (100 g) in water (1500 ml) to silica gel (500 g, 100-200 mesh)] was added, and the resulting mixture was kept at r.t. for 2 hrs. The solidified materials were filtered and washed with EtOAc. The filtrate was evaporated to dryness and the residue was purified by silica gel chromatography (petroleum ethers/EtOAc=10/1) to give a yellow solid (3 g, 38%).

### Synthesis of Intermediate 4

2N NaOH (200 ml) was added to a solution of compound 3 (3.0 g, 9.4 mmol) in EtOH (200 ml). The mixture was stirred at 60° C. for 30 min. After evaporation of the solvent, the solution was neutralized with 2N HCl to give a white precipitate. The suspension was extracted with EtOAc (2×200 ml), and the organic layers were separated, washed with water (2×100 ml), brine (2×100 ml), and dried over Na₂SO₄. Removal of the solvent gave a brown solid (2.5 g, 92%).

### Synthesis of Intermediate 6

A mixture of compound 4 (2.5 g, 8.58 mmol), compound 5 (2.52 g, 12.87 mmol), HATU (3.91 g, 10.30 mmol), and DIPEA (4.43 g, 34.32 mmol) was stirred at r.t. overnight. After the reaction mixture was filtered, the filtrate was evaporated to dryness and the residue was purified by silica gel chromatography (petroleum ethers/EtOAc=2/1) to give a brown solid (2 g, 54%).

### Synthesis of 2-(diphenylamino)-N-(7-(hydroxyamino)-7-oxoheptyl)pyrimidine-5-carboxamide (Compound A)

A mixture of the compound 6 (2.0 g, 4.6 mmol), sodium hydroxide (2N, 20 mL) in MeOH (50 ml) and DCM (25 ml) was stirred at 0° C. for 10 min. Hydroxylamine (50%) (10 ml) was cooled to 0° C. and added to the mixture. The resulting mixture was stirred at r.t. for 20 min. After removal of the solvent, the mixture was neutralized with 1M HCl to give a white precipitate. The crude product was filtered and purified by pre-HPLC to give a white solid (950 mg, 48%).

### Example 2

### Synthesis of 2-((2-chlorophenyl)(phenyl)amino)-N-(7-(hydroxyamino)-7-oxoheptyl)pyrimidine-5-carboxamide (Compound B)

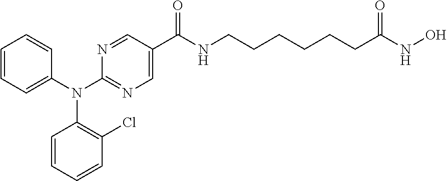 [SMILES: O=C(CCCCCCNC(=O)c1cnc(N(c2ccccc2)c2ccccc2Cl)nc1)NO]

### Reaction Scheme:

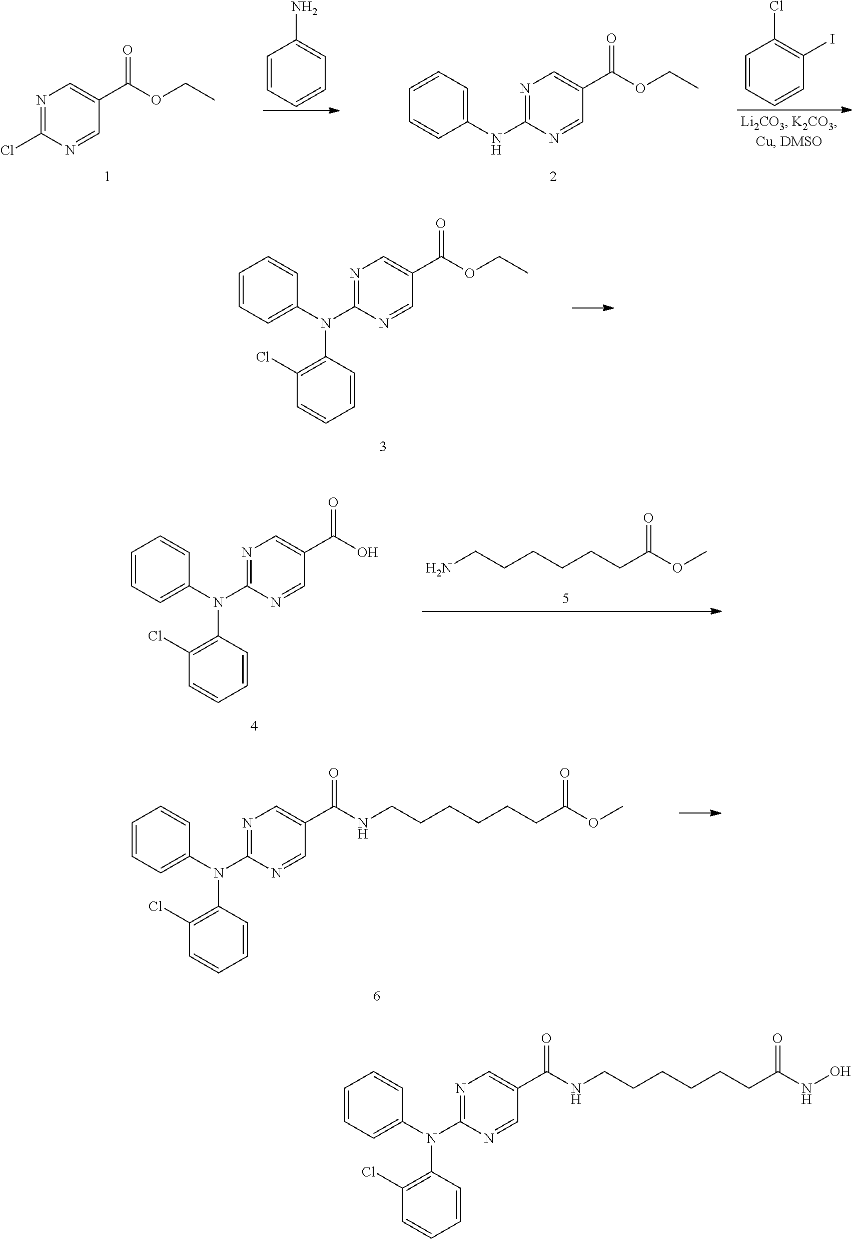

### Synthesis of Intermediate 2

See synthesis of intermediate 2 in Example 1.

### Synthesis of Intermediate 3

A mixture of compound 2 (69.2 g, 1 equiv.), 1-chloro-2-iodobenzene (135.7 g, 2 equiv.), Li₂CO₃ (42.04 g, 2 equiv.), K₂CO₃ (39.32 g, 1 equiv.), Cu (1 equiv. 45 μm) in DMSO (690 ml) was degassed and purged with nitrogen. The resulting mixture was stirred at 140° C. Work-up of the reaction gave compound 3 at 93% yield.

### Synthesis of Intermediate 4

See synthesis of intermediate 4 in Example 1.

### Synthesis of Intermediate 6

See synthesis of intermediate 6 in Example 1.

### Synthesis of 2-((2-chlorophenyl)(phenyl)amino)-N-(7-(hydroxyamino)-7-oxoheptyl)pyrimidine-5-carboxamide (Compound B)

See synthesis of Compound A in Example 1.

### Example 3

### Synthesis of 2-((1-(3-fluorophenyl)cyclohexyl)amino)-N-hydroxypyrimidine-5-carboxamide (Compound C)

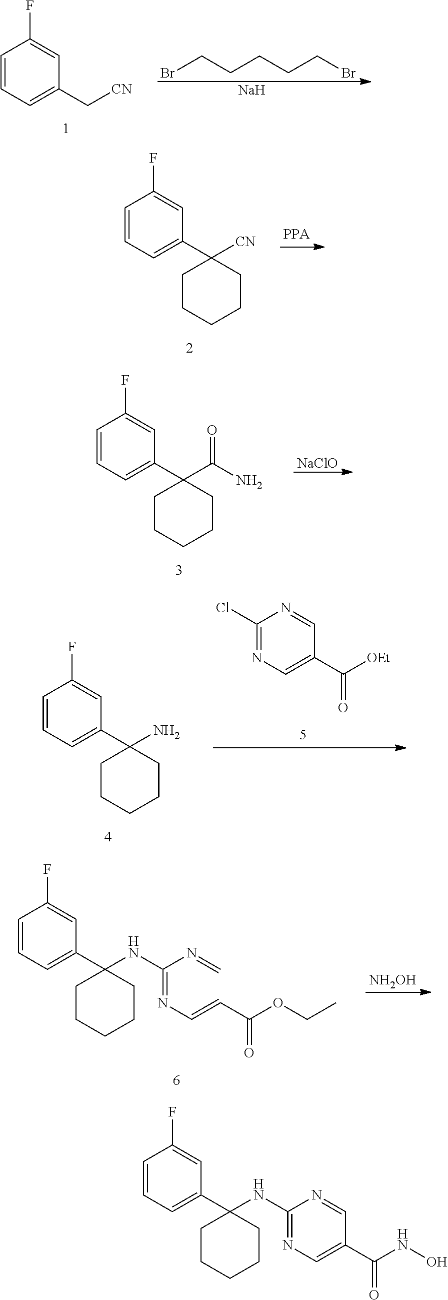

### Synthesis of Intermediate 2

To a solution of compound 1 (100 g, 0.74 mol) in dry DMF (1000 ml) was added 1,5-dibromopentane (170 g, 0.74 mol). NaH (65 g, 2.2 eq) was added dropwise while the reaction was cooled in an ice bath. The resulting mixture was vigorously stirred overnight at 50° C. The suspension was carefully quenched with ice water and extracted with ethyl acetate (3×500 ml). The combined organic layers were concentrated to afford the crude product, which was purified by flash column chromatography to give compound 2 as pale solid (100 g, 67%).

### Synthesis of Intermediate 3

A solution of compound 2 (100 g, 0.49 mol) in PPA (500 ml) was heated at 110° C. for about 5-6 hours. After completion, the resulting mixture was carefully adjusted to a pH of about 8-9 with sat.NaHCO₃ solution. The resulting precipitate was collected and washed with water (1000 ml) to afford compound 3 as white solid (95 g, 87%).

### Synthesis of Intermediate 4

To a solution of compound 3 (95 g, 0.43 mol) in n-BuOH (800 ml) was added NaClO (260 ml, 1.4 eq). 3N NaOH (400 ml, 2.8 equiv.) was then added at 0° C. and the reaction was stirred overnight at r.t. The resulting mixture was extracted with EA (2×500 ml), and the combined organic layers washed with brine. The solvent was removed in vacuo to afford the crude product which was further purified by treatment with HCl salt to yield compound 4 as a white powder (72 g, 73%).

### Synthesis of Intermediate 6

To a solution of compound 4 (2.29 g 10 mmol) in dioxane (50 ml) was added compound 5 (1.87 g, 1.0 equiv.) and DIPEA (2.58 g, 2.0 equiv.). The mixture was heated overnight at 110-120° C. The resulting mixture was directly purified on silica gel column to afford the coupled product, compound 6, as a white solid (1.37 g, 40%).

### Synthesis of 2-((1-(3-fluorophenyl)cyclohexyl)amino)-N-hydroxypyrimidine-5-carboxamide (Compound C)

To a solution of compound 6 (100 mg, 0.29 mmol) in MeOH/DCM (10 ml, 1:1) was added 50% NH₂OH in water (2 ml, excess). Sat. NaOH in MeOH (2 ml, excess) was then added at 0° C. and the reaction was stirred for 3-4 hours. After completion, the resulting mixture was concentrated and acidified with 2N HCl to reach a pH of 4-5. The precipitate was collected and washed with water (10 ml) to remove excess NH₂OH. Drying the precipitate afforded 2-((1-(3-fluorophenyl)cyclohexyl)amino)-N-hydroxypyrimidine-5-carboxamide as a white powder (70 mg, 73%).

### Example 4

### Synthesis of N-hydroxy-2-((1-phenylcyclopropyl)amino)pyrimidine-5-carboxamide (Compound D)

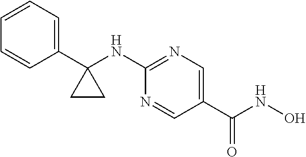 [SMILES: O=C(NO)c1cnc(NC2(c3ccccc3)CC2)nc1]

### Reaction Scheme

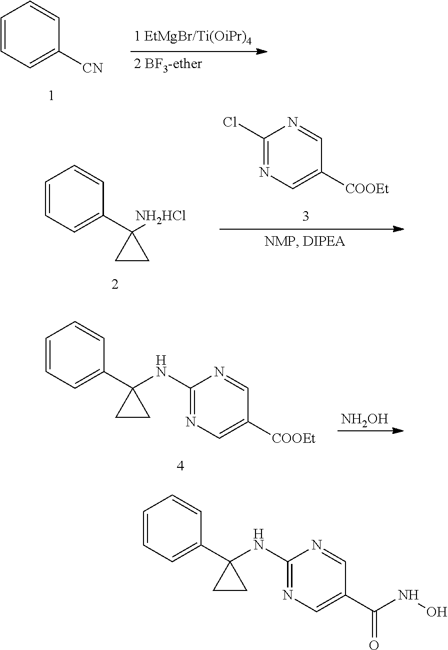

### Synthesis of Intermediate 2

A solution of compound 1, benzonitrile, (250 g, 1.0 equiv.), and Ti(OiPr)₄ (1330 ml, 1.5 equiv.) in MBTE (3750 ml) was cooled to about −10 to −5° C. under a nitrogen atmosphere. EtMgBr (1610 ml, 3.0M, 2.3 equiv.) was added dropwise over a period of 60 min., during which the inner temperature of the reaction was kept below 5° C. The reaction mixture was allowed to warm to 15-20° C. for 1 hr. BF₃-ether (1300 ml, 2.0 equiv.) was added dropwise over a period of 60 min., while the inner temperature was maintained below 15° C. The reaction mixture was stirred at 15-20° C. for 1-2 hr. and stopped when a low level of benzonitrile remained. 1N HCl (2500 ml) was added dropwise while maintaining the inner temperature below 30° C. NaOH (20%, 3000 ml) was added dropwise to bring the pH to about 9.0, while still maintaining a temperature below 30° C. The reaction mixture was extracted with MTBE (3 L×2) and EtOAc (3 L×2), and the combined organic layers were dried with anhydrous Na₂SO₄ and concentrated under reduced pressure (below 45° C.) to yield a red oil. MTBE (2500 ml) was added to the oil to give a clear solution, and upon bubbling with dry HCl gas, a solid precipitated. This solid was filtered and dried in vacuum yielding 143 g of compound 2.

### Synthesis of Intermediate 4

Compound 2 (620 g, 1.0 equiv) and DIPEA (1080 g, 2.2 equiv. were dissolved in NMP (3100 ml) and stirred for 20 min. Compound 3 (680 g, 1.02 equiv.) was added and the reaction mixture was heated to about 85-95° C. for 4 hrs. The solution was allowed to slowly cool to r.t. This solution was poured onto H₂O (20 L) and much of the solid was precipitated out from the solution with strong stirring. The mixture was filtered and the cake was dried under reduced pressure at 50° C. for 24 hr., yielding 896 g of compound 4 (solid, 86.8%).

### Synthesis of N-hydroxy-2-((1-phenylcyclopropyl)amino)pyrimidine-5-carboxamide (Compound D)

A solution of MeOH (1000 ml) was cooled to about 0-5° C. with stirring. NH₂OH HCl (1107 g, 10 equiv.) was added, followed by careful addition of NaOCH₃ (1000 g, 12.0 equiv.) The resulting mixture was stirred at 0-5° C. for one hr, and was filtered to remove the solid. Compound 4 (450 g, 1.0 equiv.) was added to the reaction mixture in one portion, and stirred at 10° C. for two hours until compound 4 was consumed. The reaction mixture was adjusted to a pH of about 8.5-9 through addition of HCl (6N), resulting in precipitation. The mixture was concentrated under reduced pressure. Water (3000 ml) was added to the residue with intense stirring and the precipitate was collected by filtration. The product was dried in an oven at 45° C. overnight (340 g, 79% yield).

### Example 5

### HDAC Enzyme Assays

Compounds for testing were diluted in DMSO to 50 fold the final concentration and a ten point three fold dilution series was made. The compounds were diluted in assay buffer (50 mM HEPES, pH 7.4, 100 mM KCl, 0.001% Tween-20, 0.05% BSA, 20 μM TCEP) to 6 fold their final concentration. The HDAC enzymes (purchased from BPS Biosciences) were diluted to 1.5 fold their final concentration in assay buffer. The tripeptide substrate and trypsin at 0.05 μM final concentration were diluted in assay buffer at 6 fold their final concentration. The final enzyme concentrations used in these assays were 3.3 ng/ml (HDAC1), 0.2 ng/ml (HDAC2), 0.08 ng/ml (HDAC3) and 2 ng/ml (HDAC6). The final substrate concentrations used were 16 μM (HDAC1), 10 μM (HDAC2), 17 μM (HDAC3) and 14 μM (HDAC6). Five μl of compound and 20 μl of enzyme were added to wells of a black, opaque 384 well plate in duplicate. Enzyme and compound were incubated together at room temperature for 10 minutes. Five μl of substrate was added to each well, the plate was shaken for 60 seconds and placed into a Victor 2 microtiter plate reader. The development of fluorescence was monitored for 60 min and the linear rate of the reaction was calculated. The IC₅₀ was determined using Graph Pad Prism by a four parameter curve fit.

### Example 6

### Inhibition of HDAC6 in a Collection of NHL Cell Lines, Both as a Single Agent and in Combination

This example describes the therapeutic potential of inhibiting HDAC6 in a collection of NHL cell lines, both as a single agent and in combination with these novel targeted agents. Treatment of lymphoma cells from a variety of molecular subtypes with selective HDAC6 inhibitors, including Compound A and the highly selective Compound C, in combination with an inhibitor of BTK resulted in synergistic decreases in lymphoma cell viability.

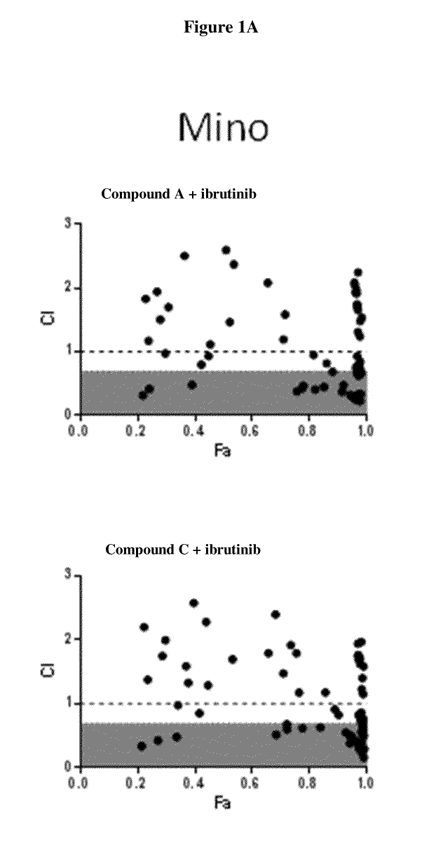

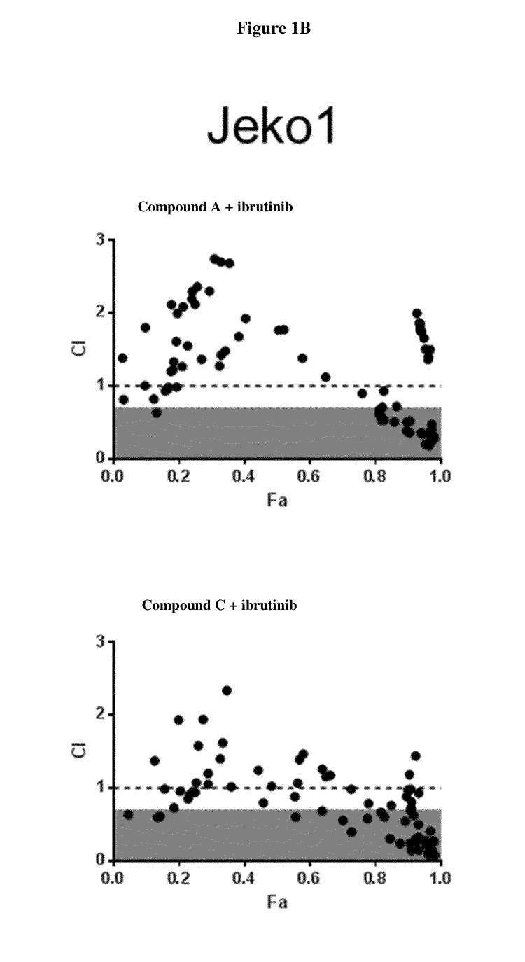

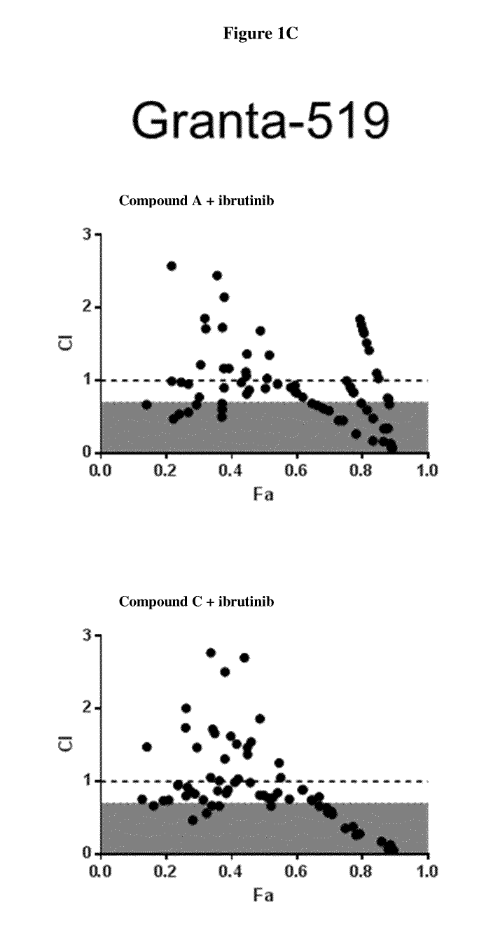

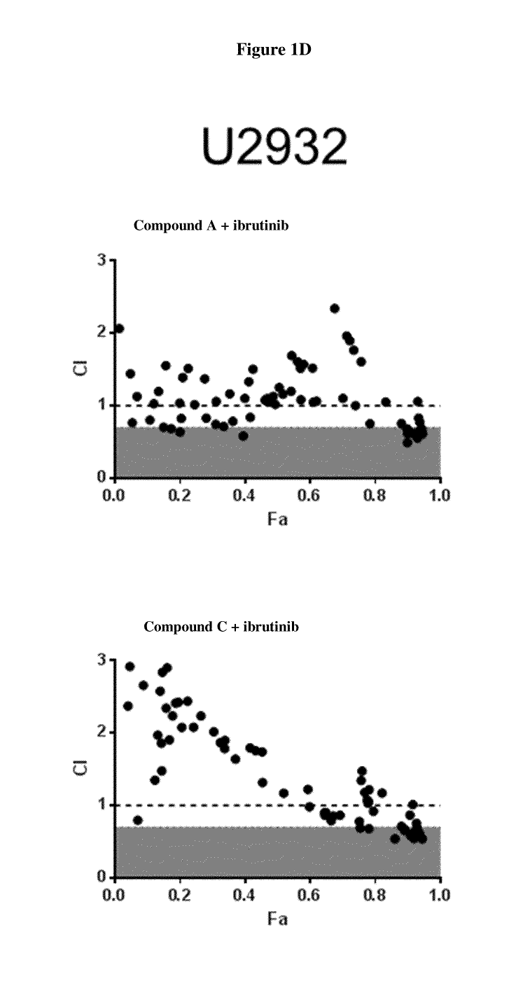

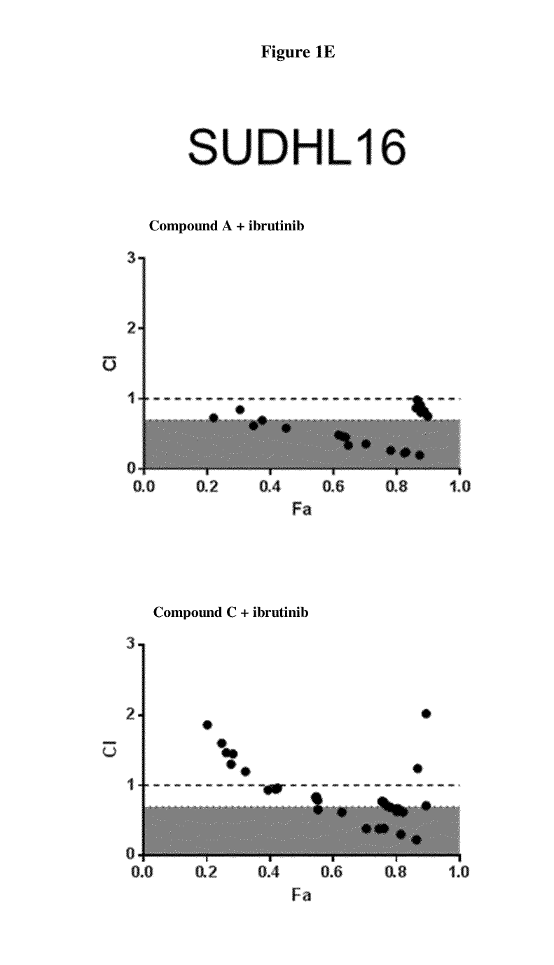

Human lymphoma cell lines were selected that represented the most common subtypes of NHL. For Mantle Cell Lymphoma (MCL) Mino, Jeko1, and Granta-519 cells were utilized, while U2932 and SUDHL16 cells represented the activated B cell (ABC) and germinal center (GC) subtypes of diffuse large B cell lymphoma (DLBCL), respectively. Briefly, cells were seeded in 384-well plates and treated in quadruplicate in a dose-matrix format with an HDAC6 inhibitor (Compound A or Compound C) in combination with a BTK inhibitor (PCI-32765/Ibrutinib). After incubating these cells for 48 hr, total cell viability was assessed via an MTS assay (Aqueous One, Promega). The fraction affected (Fa) was subsequently determined for each dose combination and the combination index (CI) was assessed using the method of Chou-Talay. CI values less than one represent a synergistic effect, values equal to one suggest an additive effect, and values greater than two indicate an antagonistic effect. As can be seen in the Fa-CI plots in FIGS. 1A-E, in all five lymphoma cell lines both HDAC6 inhibitors showed strong evidence of synergy with the tested BTK inhibitor. This is evidenced by the large number of data points (representing individual dose combinations) in the Fa-CI plot that fall below the highly stringent cutoff of 0.7. Together, these results provide strong evidence that inhibition of HDAC6 in combination with inhibition of BTK results in synergistic cell killing, and further suggests that combinations of drugs targeting both HDAC6 and BTK may provide significant clinical benefit for NHL patients.

### Example 7

### Inhibition of HDAC6 Using Compound A

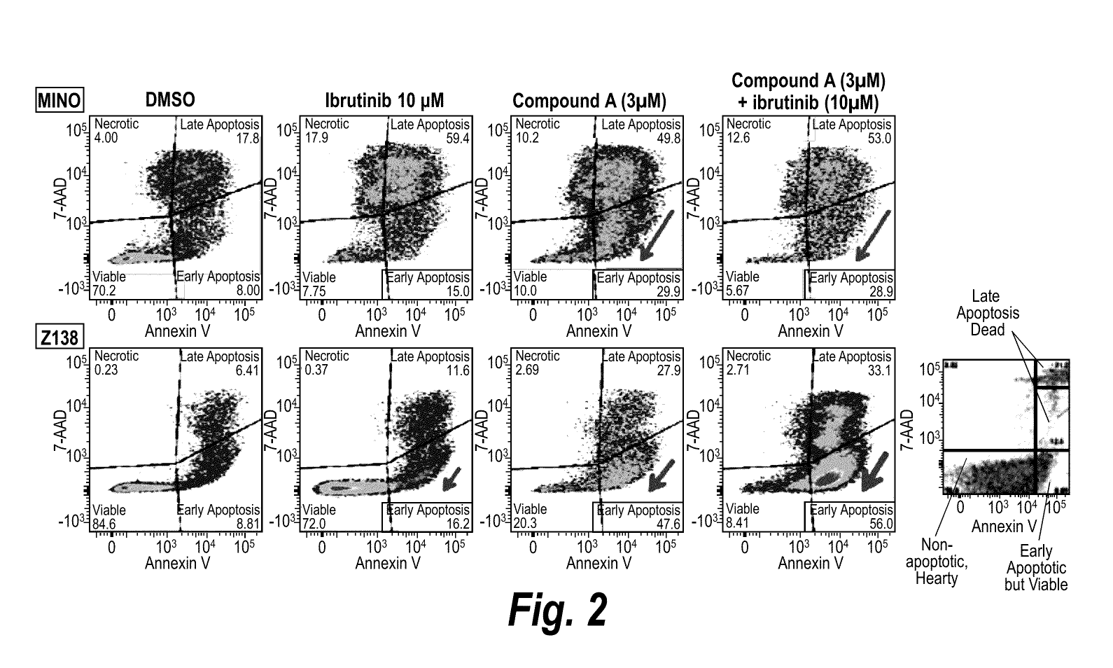

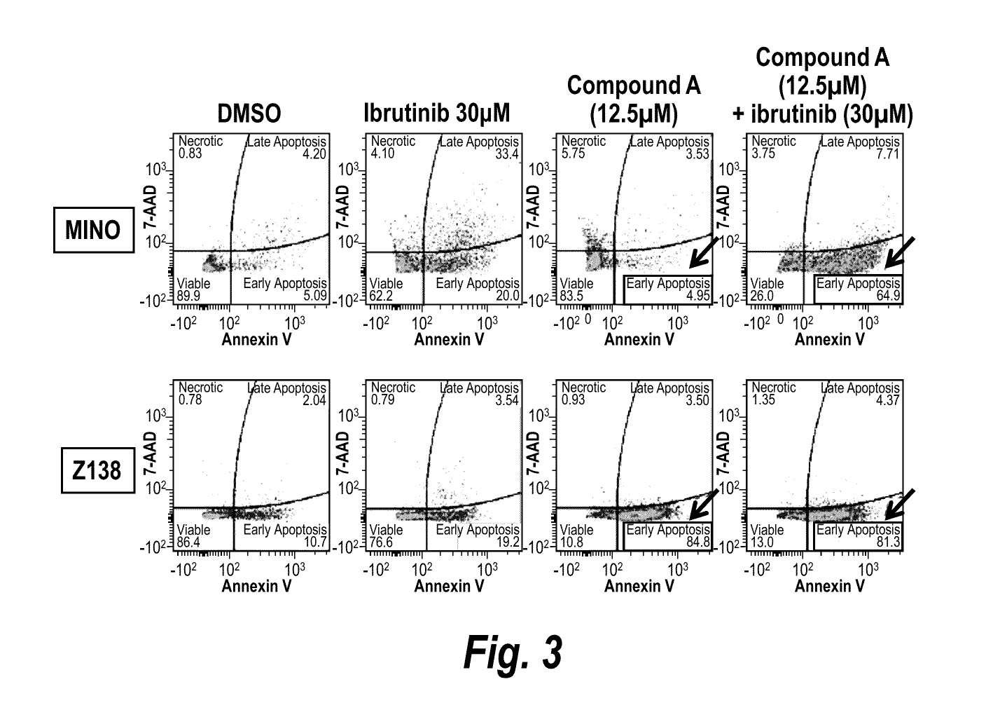

Two human MCL cell lines (Mino and Z138) were shown to have decreased viability after inhibition of HDAC6 using Compound A. Flow cytometry for the cellular markers 7-AAD and Annexin V demonstrated that treatment of both cell lines with either Compound A or ibrutinib resulted in the induction of apoptosis, which was synergistically increased by combination treatment with both drugs (FIGS. 2-3). In the experiments shown in FIG. 2, MCL MINO, and Z138 cells were treated with either Compound A (3 μM), ibrutinib (10 μM), or both compounds in combination for 48 hours. Annexin/PI flow cytometric analysis was used to determine percentage of apoptotic cells. In the experiments shown in FIG. 3, MCL MINO and Z138 cells were treated with either Compound A (12.5 μM), ibrutinib (30 μM), or both compounds in combination for 48 hours. Annexin/PI flow cytometric analysis was used to determine percentage of apoptotic cells.

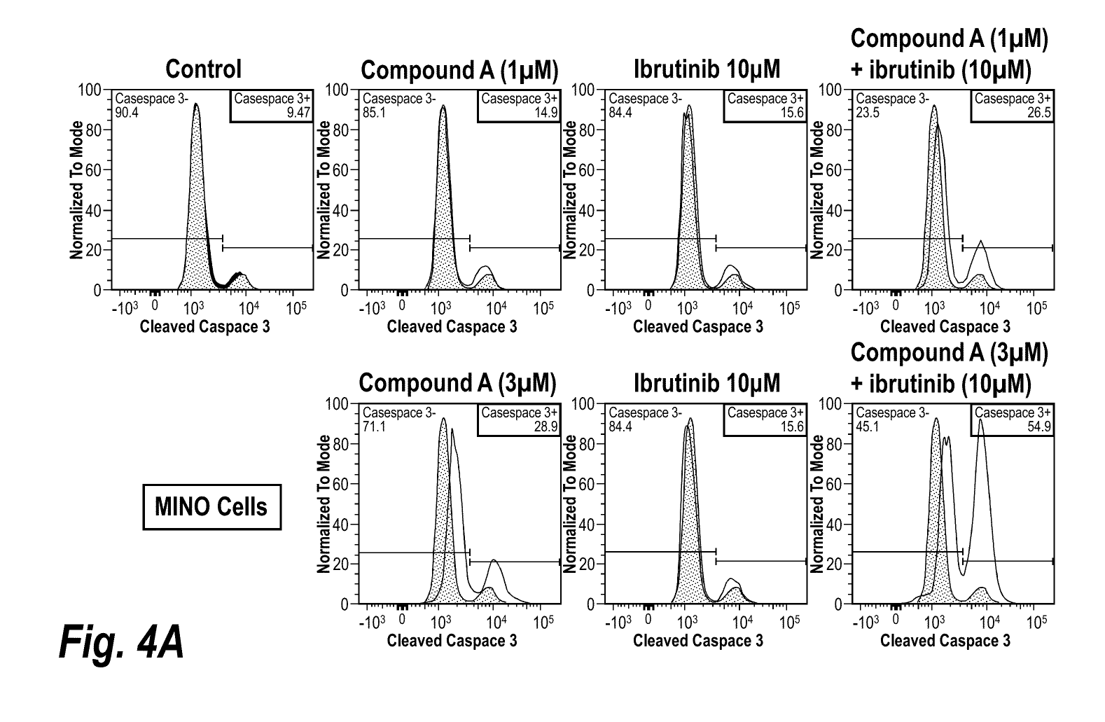

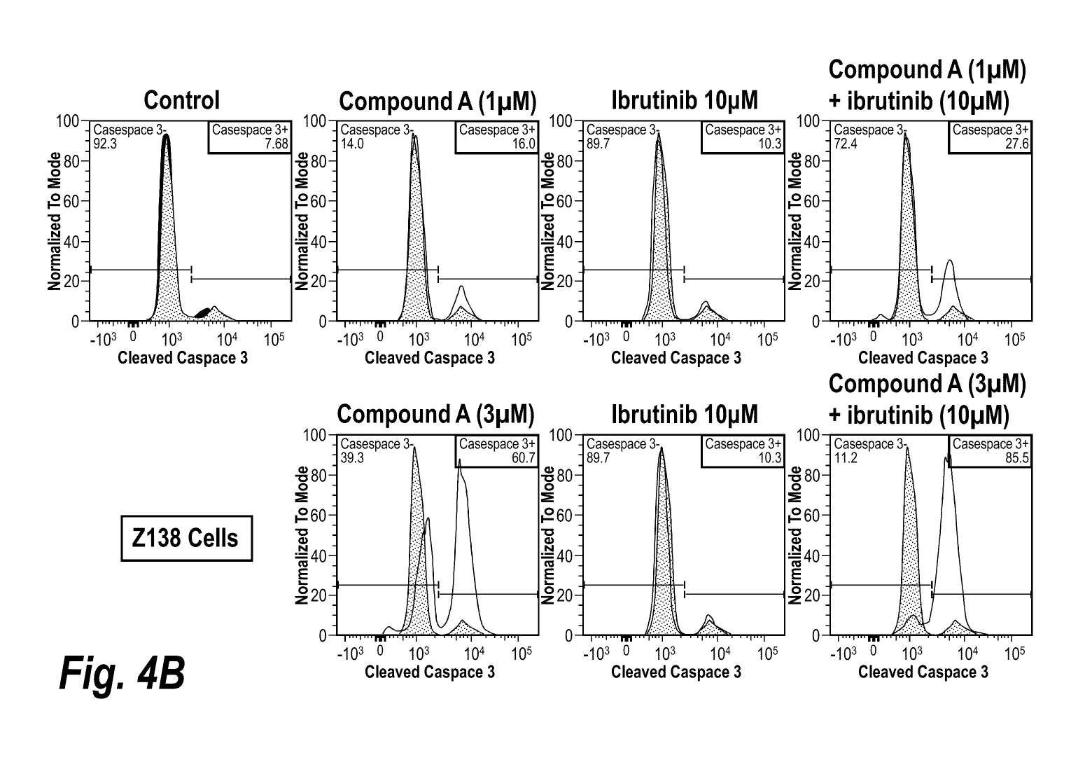

The induction of apoptosis was independently confirmed via flow cytometry for cleavage of Caspase 3 (FIG. 4). In the experiments shown in FIG. 4, MCL MINO (FIG. 4A) and Z138 (FIG. 4B) cells were treated with either Compound A (1 or 3 μM), ibrutinib (10 μM), or both compounds in combination for 48 hours. Cells were fixed and stained with activated caspase 3 antibody. Cleaved caspase 3 was detected and measured by flow cytometric analysis.

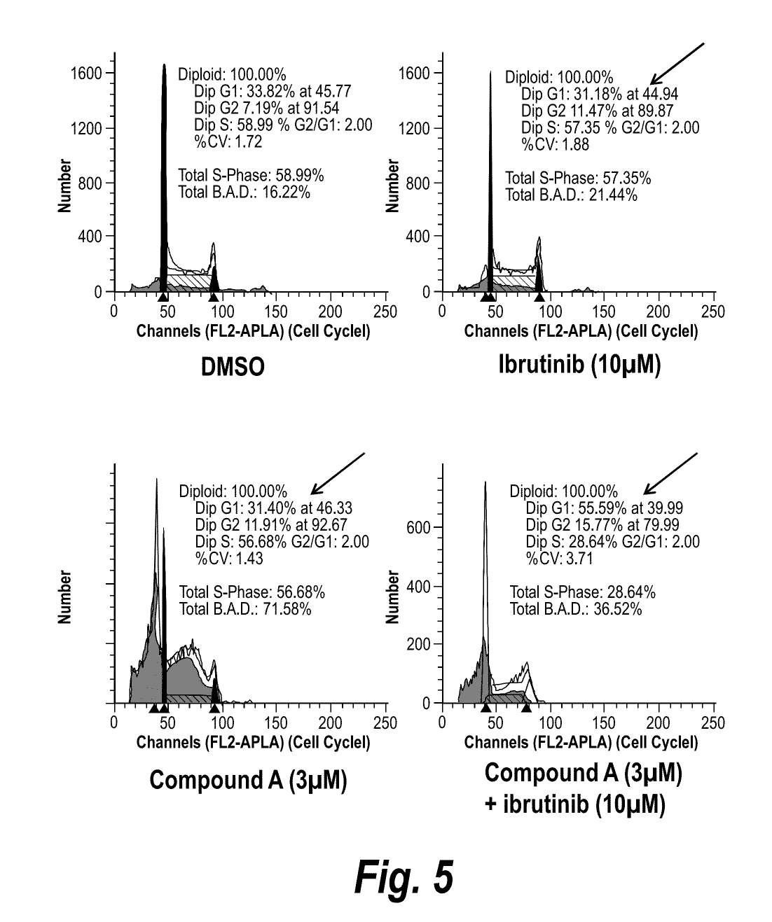

Cell cycle analysis through the incorporation of propidium iodide (PI) showed that treatment of Mino cells with either Compound A or ibrutinib had little effect on cell cycle distribution, but combination treatment with both compounds led to synergistic levels of cell cycle arrest in G1/S phase (FIG. 5). In FIG. 5, MINO cells were treated with either Compound A (13 μM), ibrutinib (10 μM), or both compounds in combination for 48 hours. Cells were fixed and stained with propidium iodide (PI) and were analyzed by flow cytometry.

These studies confirmed that combination treatment of MCL cells with an inhibitor of HDAC6 (Compound A) and an inhibitor of BTK (ibrutinib) resulted in synergistic growth arrest and induction of apoptosis.

### Example 8

### The Combination of an HDAC6 Inhibitor and a BTK Inhibitor is Well Tolerated

This example shows that the combination of an HDAC6 inhibitor and a BTK inhibitor is well tolerated in mice.

CB-17 SCID mice were treated with Vehicle, PCI-32765 (ibrutinib) alone (25 mg/kg PO QD), or PCI-32765 (25 mg/kg PO QD) plus Compound A (50 mg/kg IP QD). Percent body weight change was determined relative to the start of dosing, and the mean change±SD was plotted. All treatments were dosed five days per week for 3 cycles. All treatments were well tolerated with no overt evidence of toxicity and complete recovery after minimal body weight loss.

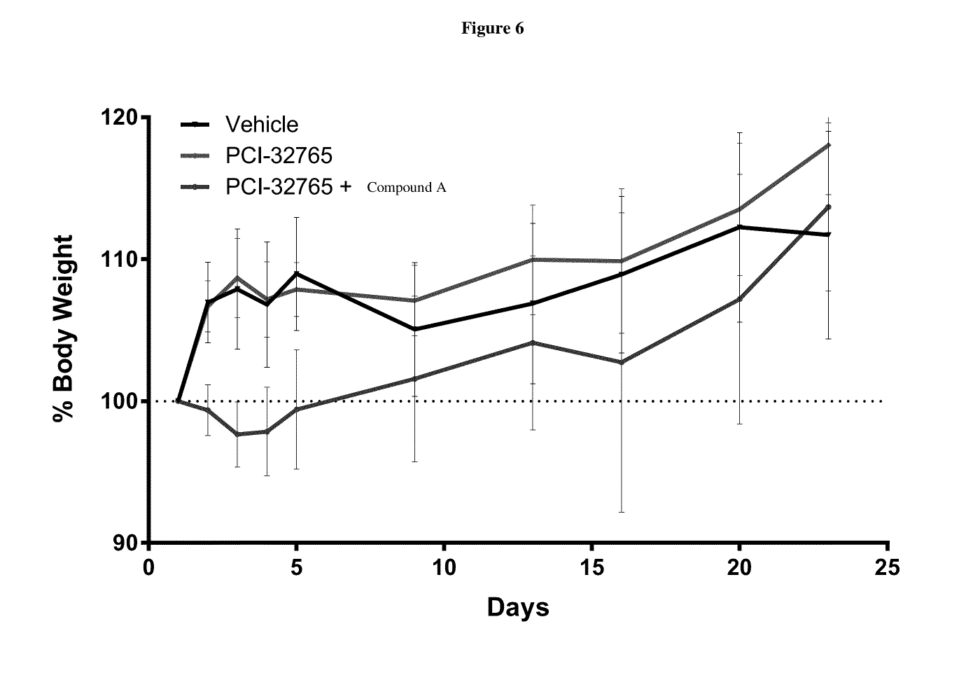

The results of these experiments are shown in FIG. 6.

### Example 9

### The Combination of an HDAC Inhibitor and a BTK Inhibitor Synergistically Decrease Viability of CLL Cells

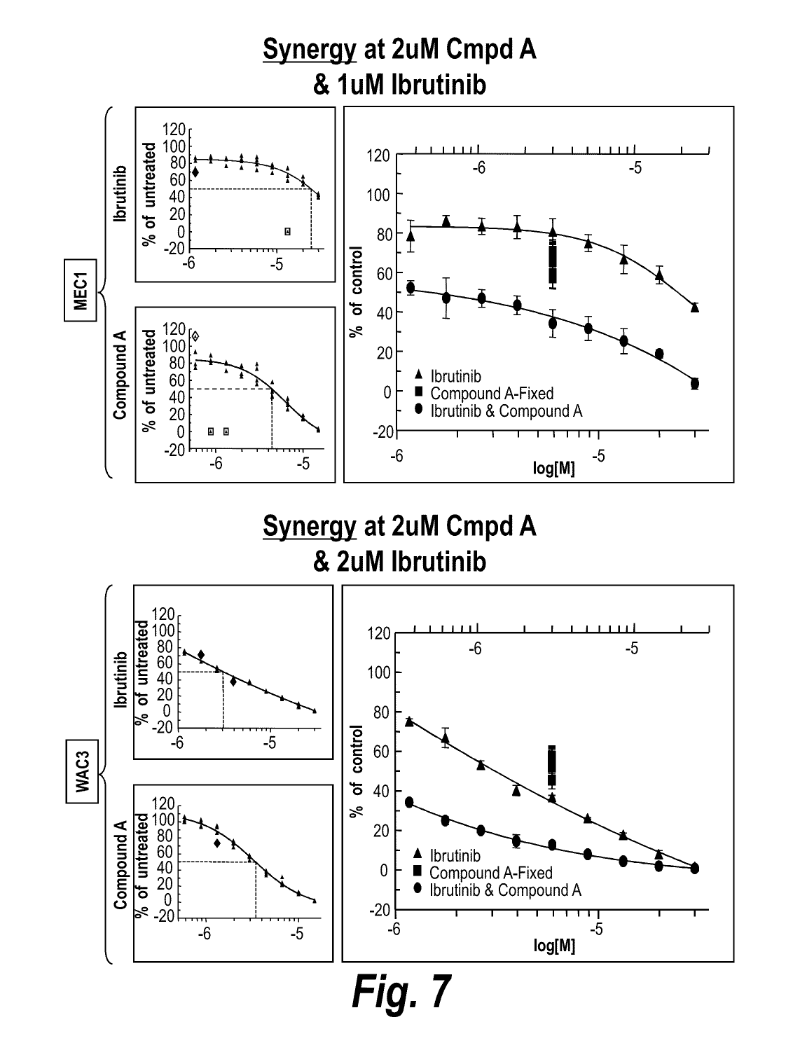

Mec1 and Wac3 CLL cells were treated with 2 μM of Compound A (Constant Concentration) and varying concentrations of the BTKi Ibrutinib for 72 hours. Cell viability was next assessed using the CellTiter-Blue reagent (Promega). The results of these experiments are shown in FIG. 7. The results demonstrate that both cell lines reach cell kill synergy with the combination of Compound A and Ibrutinib at 2 μM and 1 μM respectively.

### Example 10

### The Combination of an HDAC Inhibitor and a BTK Inhibitor Act Synergistically in MCL Cells

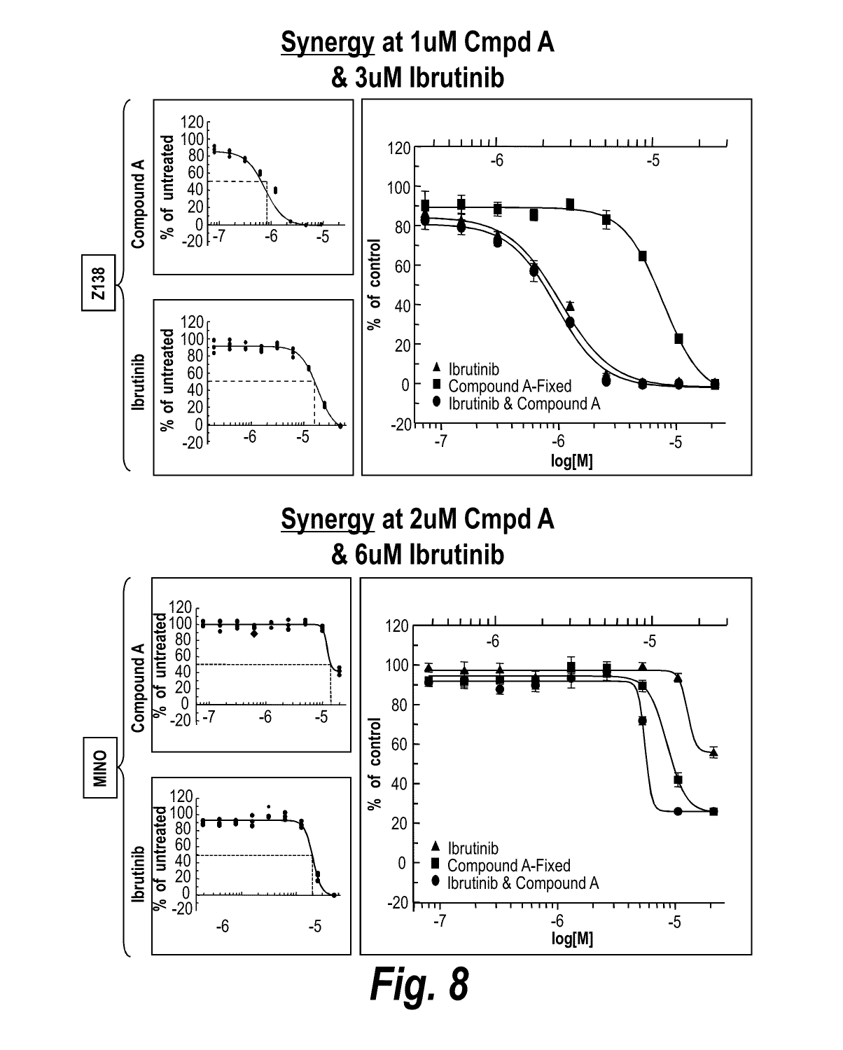

Z138 and Mino MCL cells were treated with varying concentrations of Compound A and the BTKi Ibrutinib for 72 hours. Cell viability was next assessed using the CellTiter-Blue reagent (Promega). The results of these experiments are shown in FIG. 8 and in the Table below. The results demonstrate that these cell lines reach cell kill synergy with the combination of Compound A and Ibrutinib at a 2:5 drug dilution ratio. CI values <1 indicate dose combinations resulting in synergistic decreases in cellular viability.

Experimental values (Z138)

| A = 0.858 | | B = 5.286 | | Fixed dose? | | | n | TitrateBlk | | | |
|---|---|---|---|---|---|---|---|---|---|---|---|
| Cmpd A & PCI-32765 r = 0.94 | | | | | | Cmpd A r = 0.97 | | PCI-32765 r = 0.79 | | 3 | |
| Dose | Fitted | 10\hat{ } | Ratio1 | Ratio2 | Fa | Dose | Dm | Dose | Dm | CI | log CI |
| 78.1E−9 | −0.810 | 0.15 | 0.40 | 0.00 | 0.13 | 78.1E−9 | 100.1E−9 | 195.3E−9 | 1.3E−6 | 0.932 | −0.03 |
| 156.3E−9 | −0.552 | 0.28 | 0.40 | 0.00 | 0.22 | 156.3E−9 | 202.2E−9 | 390.6E−9 | 3.3E−6 | 0.890 | −0.05 |
| 312.5E−9 | −0.294 | 0.51 | 0.40 | 0.00 | 0.34 | 312.5E−9 | 408.3E−9 | 781.2E−9 | 8.6E−6 | 0.857 | −0.07 |
| 625.0E−9 | −0.036 | 0.92 | 0.40 | 0.00 | 0.48 | 625.0E−9 | 824.5E−9 | 1.6E−6 | 22.0E−6 | 0.829 | −0.08 |
| 1.3E−6 | 0.223 | 1.67 | 0.40 | 0.00 | 0.63 | 1.3E−6 | 1.7E−6 | 3.1E−6 | 56.7E−6 | 0.806 | −0.09 |
| 2.5E−6 | 0.481 | 3.03 | 0.40 | 0.00 | 0.75 | 2.5E−6 | 3.4E−6 | 6.2E−6 | 146.1E−6 | 0.786 | −0.10 |
| 5.0E−6 | 0.739 | 5.48 | 0.40 | 0.00 | 0.85 | 5.0E−6 | 6.8E−6 | 12.5E−6 | 376.3E−6 | 0.770 | −0.11 |
| 10.0E−6 | 0.997 | 9.94 | 0.40 | 0.00 | 0.91 | 10.0E−6 | 13.7E−6 | 25.0E−6 | 969.0E−6 | 0.755 | −0.12 |
| 20.0E−6 | 1.256 | 18.01 | 0.40 | 0.00 | 0.95 | 20.0E−6 | 27.7E−6 | 50.0E−6 | 2.5E−3 | 0.742 | −0.13 |

Experimental values (Mino)

| A = 4.007 | | B = 21.892 | | Fixed dose? | | | n | TitrateBlk | | | |
|---|---|---|---|---|---|---|---|---|---|---|---|
| Cmpd A & PCI-327655 r = 0.91 | | | | | | Cmpd A r = 0.7 | | PCI-32765 r = 0.92 | | 3 | |
| Dose | Fitted | 10\hat{ } | Ratio1 | Ratio2 | Fa | Dose | Dm | Dose | Dm | CI | log CI |
| 78.1E−9 | −6.589 | 0.00 | 0.40 | 0.00 | 0.00 | 78.1E−9 | 338.3E−12 | 195.3E−9 | 308.7E−9 | 231.587 | 2.36 |
| 156.3E−9 | −5.383 | 0.00 | 0.40 | 0.00 | 0.00 | 156.3E−9 | 2.8E−9 | 390.6E−9 | 621.3E−9 | 56.491 | 1.75 |
| 312.5E−9 | −4.176 | 0.00 | 0.40 | 0.00 | 0.00 | 312.5E−9 | 23.1E−9 | 781.2E−9 | 1.3E−6 | 14.137 | 1.15 |
| 625.0E−9 | −2.970 | 0.00 | 0.40 | 0.00 | 0.00 | 625.0E−9 | 191.2E−9 | 1.6E−6 | 2.5E−6 | 3.889 | 0.59 |
| 1.3E−6 | −1.764 | 0.02 | 0.40 | 0.00 | 0.02 | 1.3E−6 | 1.6E−6 | 3.1E−6 | 5.1E−6 | 1.408 | 0.15 |
| 2.5E−6 | −0.557 | 0.28 | 0.40 | 0.00 | 0.22 | 2.5E−6 | 13.1E−6 | 6.2E−6 | 10.2E−6 | 0.805 | −0.09 |
| 5.0E−6 | 0.649 | 4.46 | 0.40 | 0.00 | 0.82 | 5.0E−6 | 108.1E−6 | 12.5E−6 | 20.5E−6 | 0.656 | −0.18 |
| 10.0E−6 | 1.855 | 71.67 | 0.40 | 0.00 | 0.99 | 10.0E−6 | 894.0E−6 | 25.0E−6 | 41.3E−6 | 0.617 | −0.21 |
| 20.0E−6 | 3.062 | #### | 0.40 | 0.00 | 1.00 | 20.0E−6 | 7.4E−3 | 50.0E−6 | 83.0E−6 | 0.605 | −0.22 |

### INCORPORATION BY REFERENCE

The contents of all references (including literature references, issued patents, published patent applications, and co-pending patent applications) cited throughout this application are hereby expressly incorporated herein in their entireties. Unless otherwise defined, all technical and scientific terms used herein are accorded the meaning commonly known to one with ordinary skill in the art.

### EQUIVALENTS

Those skilled in the art will recognize, or be able to ascertain using no more than routine experimentation, many equivalents of the specific embodiments of the invention described herein. Such equivalents are intended to be encompassed by the following claims.

What is claimed is:

1. A method for treating non-hodgkin's lymphoma in a subject in need thereof comprising administering to the subject a therapeutically effective amount of a histone deacetylase 6 (HDAC6) specific inhibitor.

2. The method of claim 1, wherein the HDAC6 specific inhibitor is a compound of Formula I: 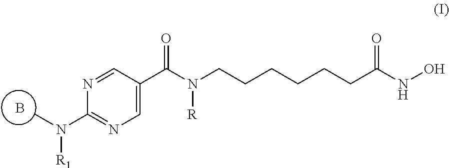
or a pharmaceutically acceptable salt thereof,
wherein,
ring B is aryl or heteroaryl;
R₁ is an aryl or heteroaryl, each of which may be optionally substituted by OH, halo, or C₁₋₆-alkyl;
and
R is H or C₁₋₆-alkyl.

3. The method of claim 2, wherein the compound of Formula I is: 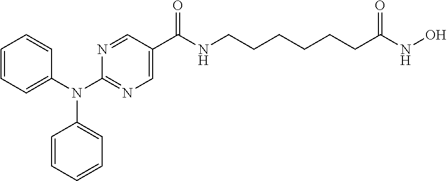 [SMILES: O=C(CCCCCCNC(=O)c1cnc(N(c2ccccc2)c2ccccc2)nc1)NO]
or a pharmaceutically acceptable salt thereof.

4. The method of claim 2, wherein the compound of Formula I is: 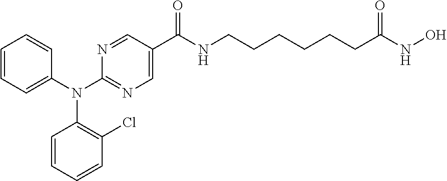 [SMILES: O=C(CCCCCCNC(=O)c1cnc(N(c2ccccc2)c2ccccc2Cl)nc1)NO]
or a pharmaceutically acceptable salt thereof.

5. The method of claim 1, wherein the HDAC6 specific inhibitor is a compound of Formula II: 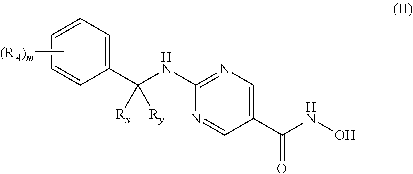
or a pharmaceutically acceptable salt thereof,
wherein,
Rₓ and R_{y} together with the carbon to which each is attached, form a cyclopropyl, cyclobutyl, cyclopentyl, cyclohexyl, cycloheptyl, or cyclooctyl;
each R_{A} is independently C₁₋₆-alkyl, C₁₋₆-alkoxy, halo, OH, —NO₂, —CN, or —NH₂; and
m is 0, 1, or 2.

6. The method of claim 5, wherein the compound of Formula II is: 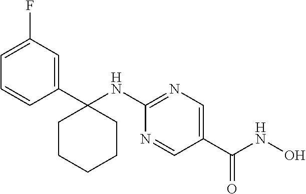 [SMILES: O=C(NO)c1cnc(NC2(c3cccc(F)c3)CCCCC2)nc1]
or a pharmaceutically acceptable salt thereof.

7. The method of claim 5, wherein the compound of Formula II is: 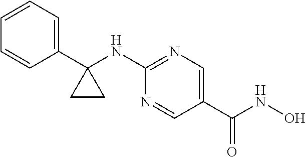 [SMILES: O=C(NO)c1cnc(NC2(c3ccccc3)CC2)nc1]
or a pharmaceutically acceptable salt thereof.

8. The method of claim 1, wherein the method further comprises administering to the subject a therapeutically effective amount of a Bruton's tyrosine kinase (BTK) inhibitor.

9. The method of claim 8, wherein the BTK inhibitor is ibrutinib or a pharmaceutically acceptable salt thereof.

10. The method of claim 8, wherein the HDAC6 specific inhibitor is administered at a sub-therapeutic dose.

11. The method of claim 1, wherein the HDAC6 specific inhibitor induces apoptosis of cancer cells.

12. A pharmaceutical combination for treating non-hodgkin's lymphoma comprising a therapeutically effective amount of a histone deacetylase 6 (HDAC6) specific inhibitor or a pharmaceutically acceptable salt thereof, and a Bruton's tyrosine kinase (BTK) inhibitor or a pharmaceutically acceptable salt thereof.

13. The combination of claim 12, wherein the HDAC6 specific inhibitor is a compound of Formula I: 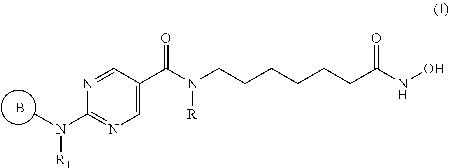
or a pharmaceutically acceptable salt thereof,
wherein,
ring B is aryl or heteroaryl;
R₁ is an aryl or heteroaryl, each of which may be optionally substituted by OH, halo, or C₁₋₆-alkyl;
and
R is H or C₁₋₆-alkyl.

14. The combination of claim 13, wherein the compound of Formula I is: 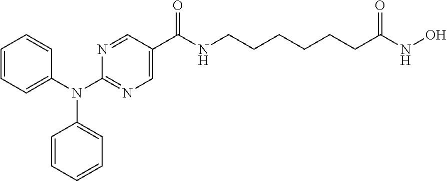 [SMILES: O=C(CCCCCCNC(=O)c1cnc(N(c2ccccc2)c2ccccc2)nc1)NO]
or a pharmaceutically acceptable salt thereof.

15. The combination of claim 13, wherein the compound of Formula I is: 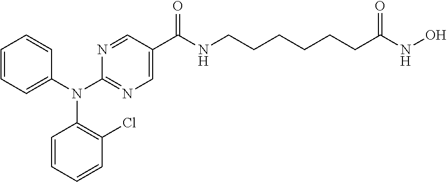 [SMILES: O=C(CCCCCCNC(=O)c1cnc(N(c2ccccc2)c2ccccc2Cl)nc1)NO]
or a pharmaceutically acceptable salt thereof.

16. The combination of claim 12, wherein the HDAC6 specific inhibitor is a compound of Formula II: 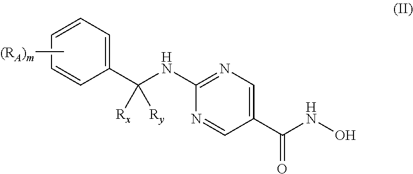
or a pharmaceutically acceptable salt thereof,
wherein,
Rₓ and R_{y} together with the carbon to which each is attached, form a cyclopropyl, cyclobutyl, cyclopentyl, cyclohexyl, cycloheptyl, or cyclooctyl;
each R_{A} is independently C₁₋₆-alkyl, C₁₋₆-alkoxy, halo, OH, —NO₂, —CN, or —NH₂; and
m is 0, 1, or 2.

17. The combination of claim 16, wherein the compound of Formula II is: 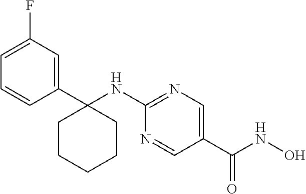 [SMILES: O=C(NO)c1cnc(NC2(c3cccc(F)c3)CCCCC2)nc1]
or a pharmaceutically acceptable salt thereof.

18. The combination of claim 16, wherein the compound of Formula II is: 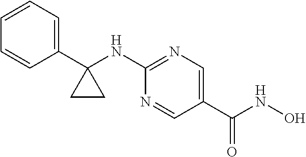 [SMILES: O=C(NO)c1cnc(NC2(c3ccccc3)CC2)nc1]
or a pharmaceutically acceptable salt thereof.

19. The combination of claim 12, wherein the BTK inhibitor is ibrutinib or a pharmaceutically acceptable salt thereof.

20. The combination of claim 12, wherein the combination further comprises a pharmaceutically acceptable carrier.

21. A method for decreasing cell viability of cancer cells by administering an HDAC inhibitor, or a combination comprising a histone deacetylase (HDAC) inhibitor and a Bruton's tyrosine kinase (BTK) inhibitor.

22. A method for synergistically increasing apoptosis of cancer cells by administering a combination comprising a histone deacetylase (HDAC) inhibitor and a Bruton's tyrosine kinase (BTK) inhibitor.

23. A method for synergistically increasing cleavage of caspase 3 in cancer cells by administering a combination comprising a histone deacetylase (HDAC) inhibitor and a Bruton's tyrosine kinase (BTK) inhibitor.

24. A method for synergistically arresting cells in the G1/S phase of the cell cycle by administering a combination comprising a histone deacetylase (HDAC) inhibitor and a Bruton's tyrosine kinase (BTK) inhibitor.
